(19) United States
(12) Patent Application Publication
(10) Pub. No.: US 2005/0136449 A1
(43) Pub. Date: Jun. 23, 2005

(54) Compositions and methods for synthesizing, purifying, and detecting biomolecules

(75) Inventors: George Hanson, Madison, WI (US); Wieslaw Antoni Kudlicki, Carlsbad, CA (US); Shiranthi Keppetipola, Vista, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation, Carlsbad, CA (US)

(21) Appl. No.: 10/954,951

(22) Filed: Oct. 1, 2004

Related U.S. Application Data
(60) Provisional application No. 60/508,142, filed on Oct. 1, 2003.

(51) Int. Cl. C12Q 1/68; G01N 33/554; G01N 33/569; C12N 9/52

(52) U.S. Cl. 435/6; 435/220; 435/252.33; 435/7.32; 530/400

(57) ABSTRACT

Compositions and methods that can reduce or eliminate purification and detection difficulties related to SlyD polypeptides are disclosed. The compositions include cells that lack or contain a reduced amount of SlyD, cells that contain SlyD mutated to reduce or eliminate its ability to bind biarsenical reagents; anti-SlyD antibodies, and kits containing the same. The disclosed compositions can be used, e.g., to purify and detect recombinant polypeptides having polyhistidine or polycysteine tags.

### CROSS-REFERENCE TO RELATED APPLICATION

The present invention claims the benefit of U.S. Provisional Application No. 60/508,142, filed Oct. 1, 2003, the disclosure of which is incorporated herein by reference in its entirety.

### BACKGROUND OF THE INVENTION

1. Field of the Invention

This invention relates to the biotechnology field. In particular, the invention relates to in vitro systems for synthesizing, purifying and/or detecting biomolecules, such as nucleic acids and polypeptides.

2. Related Art

Synthesizing, purifying and detecting biomolecules (e.g., polypeptides and nucleic acids) is an important aspect of biotechnology research, development and production. Biomolecules typically are made in cell culture (e.g., using host cells containing a recombinant nucleic acid that can give rise to a desired recombinant polypeptide). Purifying a desired biomolecule from cell culture growth medium, products of cellular metabolism, and/or other cellular constituents to a degree suitable for research, diagnostic, therapeutic or medical purposes can be a time-consuming and/or problematic process. Detecting a particular biomolecule in mixtures that include cell culture growth medium, products of cellular metabolism, and/or other cellular constituents also can be time-consuming and/or problematic process.

### BRIEF SUMMARY OF THE INVENTION

Host cell polypeptides can interfere with the synthesis, purification and/or detection of desired biomolecules (e.g., recombinant polypeptides).

In a first aspect, compositions and methods that can reduce or eliminate purification and detection difficulties caused by the SlyD protein, which is encoded by the slyD gene, are provided.

In a second aspect, compositions (including cellular extracts) that can be used for biomolecule synthesis, that are or are derived from a host cell that has been engineered or. manipulated so as to eliminate or reduce the amount of SlyD, are provided.

In a third aspect, compositions and methods of the invention involve a host cell engineered to contain a SlyD polypeptide that has been mutated to reduce or eliminate its ability to bind biarsenical reagents, are provided.

In a fourth aspect, compositions and methods of the invention involve anti-SlyD antibodies that can specifically remove SlyD from a mixture containing desired biomolecule(s), are provided.

In a fifth aspect, the invention provides an in vitro protein synthesis (IVPS) composition that is (a) prepared from an organism or cell that has been manipulated or engineered to be depleted in SlyD ptotein; (b) supplemented with a composition comprising one or more detergents; or (c) combinations of one or more of (a), (b) and (c).

In another aspect, kits comprising one or more compositions of the invention are provided. In one such aspect, the kits of the invention comprise one or more of the following: (a) one or more host cells, said host cells having a mutation (in certain embodiments, a deletion mutation) in a slyD gene; (b) one or more nucleic acids having a sequence that is the reverse complement of an endogenous slyD gene, which may be selected from the group consisting of an antisense oligonucleotide and an an RNAi molecule; and (c) one or more molecules that specifically binds to a SlyD polypeptide, which may be an antibody. In certain such aspects, the one or more host cells may be one or more bacterial cells, including but not limited to an E. coli strain, such as E. coli strains JDP670, JDP671, JDP687, JDP689, JDP694, JDP704, JDP707, RY7425 (CQ21 zac::Tn10kan zhd::Tn10) slyD1, and A19 slyD::kan, and preferably E. coli strain JDP89. In certain such kits of the invention, the host cells are lyophilized.

Certain such kits of the invention may further comprise an arsenical molecule, such as a biarsenical molecule including, but not limited to, EDT₂[4′,5′-bis(1,3,2-dithioarsolan-2-yl)fluorescein-(1,2-ethanedithiol)₂]. In certain such aspects, the arsenical and/or biarsenical molecules are detectably labeled.

In additional embodiments, the kits of the invention may further comprise one or more nucleic acids, which may be selected from the group consisting of a control DNA molecule, a cloning vector and an expression vector.

In additional embodiments, the kits of the invention may further comprise one or more enzymes, which may be selected from the group consisting of a restriction endonuclease, a nucleic acid polymerase, a nucleic acid ligase, a nucleic acid topoisomerase, a site-specific DNA recombinase, a uracil DNA glycosylase, a protease, a phosphatase, a ribonuclease and a ribonuclease inhibitor.

In additional embodiments, the kits of the invention may further comprise one or more transfection reagents and/or one or more growth media.

In additional embodiments, the invention provides kits comprising one or more molecules that specifically bind SlyD. In certain such aspects, one or more of the molecules that specifically bind SlyD is an antibody. Additional such kits of the invention may further comprise a nickel resin, and/or may further comprise a solid substrate attached to or coated with EDT₂[4′,5′-bis(1,3,2-dithioarsolan-2-yl)fluorescein-(1,2-ethanedithiol)]₂.

In another aspect, methods for synthesizing, purifying or detecting biomolecules, are provided. Certain such aspects provide methods of purifying a protein comprising a tag, such as a polyhistidine tag or a tetra-Cys tag, from a solution by MIAC, comprising contacting said solution with a molecule that specifically binds SlyD such as an antibody.

Unless otherwise defined, all technical and scientific terms used herein have the meaning commonly understood by one skilled in the biotechnology art. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description.

### BRIEF DESCRIPTION OF THE DRAWINGS

FIG. 1: Model of a Lumio™ Reagent binding a tetracysteine motif.

FIG. 2: Biarsenical (FlAsH-EDT₂ or ReAsH-EDT₂) labeling of several versions of SlyD. Cell extracts from in vitro protein synthesis reactions were labeled with F1AsH-EDT₂ and separated by SDS-PAGE. Lane 1 is full length, hexahistidine tagged SlyD (SlyD+His tag), Lane 2 is full length, hexahistidine tagged SlyD with two point mutations: C167A and C168A (SlyD-C167A/C168A), and Lane 3 contains a hexahistidine tagged version of SlyD truncated after position 171 (SlyD-truncl71).

FIG. 3: Gel imaged with A) Typhoon 8600 Variable mode Imager, B) standard UV light box, and C) white-light for total protein profile with Coomassieg blue stain. Lanes 1-3: CAT; Lanes 4-6: GFP; Lanes 7-9: GUS; Lane 10: no DNA control reaction. D) Analysis of the sensitivity of Lumio™ Green Detection Reagent using purified Adenylate Kinase 1. CAT, GFP, and GUS were expressed from pEXP3-DEST plasmid.

FIG. 4: A) Real-time incorporation of Lumio™ to in vitro synthesized proteins. These proteins were expressed from the plasmid: pRSETb. B) In-gel detection of expressed proteins; ACP (Acyl carrier protein with C-terminal CCPGCC), AcpS (Acyl carrier protein S protein with C-terminal CCGGKGNGGCGC), CaM (Calmodulin with N-terminal CCEQCC), CaM Ortho (Calmodulin with N-terminal CCEQCC and C-terminal CGPCCGPC), and SlyD (full-length SlyD with naturally occurring C-terminal CCGGKGNGGCGC).

FIG. 5: A) In-gel detection of expressed proteins. Lane 1: v-crk avian sarcoma virus, 2: cAMP-dependent protein kinase, 3: adenylate kinase, 4: creatine kinase 5: no DNA control. B) Real-time incorporation of Lumio™ Green Detection Reagent into in vitro synthesized proteins.

### DETAILED DESCRIPTION OF THE INVENTION

I. Defintions

In the description that follows, a number of terms used in recombinant nucleic acid technology are utilized extensively. In order to provide a clear and more consistent understanding of the specification and claims, including the scope to be given such terms, the following definitions are provided.

Arsenical Molecule:

As used herein, an arsenical molecule is any chemical compound comprising one or more atoms of Arsenic. Preferred arsenical molecules bind a specific amino acid sequence. A preferred specific amino acid sequence is C-C-X-X-C-C, wherein “C” represents cysteine and “X” represents any amino acid other than cysteine. Both biarsenical (2 arsenic atoms) and tetraarsenical (4 arsenic atoms) compounds are arsenical compounds. A tetraarsenical molecule is both an arsenical and biarsenical molecule.

An arsenical, biarsenical or tetraarsenical molecule preferably includes a detectable group, for example a fluorescent group, a luminescent group, a phosphorescent group, a spin label, a photosensitizer, a photocleavable moiety, a chelating center, a heavy atom, a radioactive isotope, an isotope detectable by nuclear magnetic resonance (NMR), a paramagnetic atom, and combinations thereof. For some applications, the biarsenical molecule is immobilized on a solid phase, preferably by covalent coupling. Such applications include being immobilized on beads or some other substrate suitable for affinity chromatography. This is used to purify tagged proteins. An arsenical, biarsenical or tetraarsenical molecule preferably is capable of traversing a biological membrane.

Biarsenical Molecule:

As used herein a biarsenical molecule is any chemical compound comprising two or more atoms of Arsenic. Preferred biarsenical molecules bind a specific amino acid sequence. A preferred specific amino acid sequence is C-C-X-X-C-C, wherein “C” represents cysteine and “X” represents any amino acid other than cysteine.

Tetraarsenical Molecule:

Other molecules that can used instead of or in combination with a biarsenical molecule include without limitation a tetraarsenical molecule. The tetraarsenical molecule includes two biarsenical molecules having chemical formulas disclosed in U.S. Pat. No. 6,054,271 to Tsien. For example, two biarsenical molecules are coupled to each other through a linking group.

Detectably Labeled:

The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection means, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, radioactive isotope labels, fluorescent labels, chemiluminescent labels, bioluminescent labels and enzyme labels, and others that will be familiar to those of ordinary skill in the art.

Gene:

As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Host:

As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

IVT:

The terms “in vitro transcription” (IVT) and “cell-free transcription” are used interchangeably herein and are intended to refer to any method for cell-free synthesis of RNA from DNA without synthesis of protein from the RNA. A preferred RNA is messenger RNA (mRNA), which encodes proteins.

IVTT:

The terms “in vitro transcription-translation” (IVTT), “cell-free transcription-translation”, “DNA template-driven in vitro protein synthesis” and “DNA template-driven cell-free protein synthesis” are used interchangeably herein and are intended to refer to any method for cell-free synthesis of mRNA from DNA (transcription) and of protein from mRNA (translation).

IVPS:

The terms “in vitro protein synthesis” (IVPS), “in vitro translation”, “cell-free translation”, “RNA template-driven in vitro protein synthesis”, “RNA template-driven cell-free protein synthesis” and “cell-free protein synthesis” are used interchangeably herein and are intended to refer to any method for cell-free synthesis of a protein. IVTT is one non-limiting example of IVPS.

Nucleic Acid Molecule:

As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Polypeptide:

As used herein, the term “polypeptide” refers to a sequence of contiguous amino acids of any length. The terms “peptide,” “oligopeptide,” or “protein” may be used interchangeably herein with the term “polypeptide.”

Other terms used in the fields of recombinant nucleic acid technology and molecular and cell biology as used herein will be generally understood by one of ordinary skill in the applicable arts.

II. Overview

Host cell polypeptides can interfere with the purification of desired biomolecules. Such interference can occur when a desired polypeptide and a host cell polypeptide behave similarly or identically during one or more purification steps. For example, when a desired polypeptide and a host cell polypeptide include motifs that can interact with a separation medium during a chromatographic purification step, the host cell polypeptide can co-purify as a contaminant with the desired polypeptide.

Host cell polypeptides also can interfere with the detection of desired biomolecules. For example, when a desired polypeptide and a host cell polypeptide interact with a detection reagent, labeling of the host cell polypeptide by the detection reagent can create background that interferes with the detection of the desired polypeptide.

The disclosed inventions are based in part on the surprising finding that the E. coli SlyD polypeptide can interact with biarsenical reagents. SlyD can interact with biarsenical purification reagents, including those used in biarsenical immobilized metal affinity chromatography (IMAC). In biarsenical IMAC, recombinant polycysteine-tagged (Cys-tagged) polypeptides interact with and are selectively retained in association with a biarsenical-containing separation medium, allowing them to be purified from a mixture. Problematically, SlyD also can interact with a biarsenical-containing separation medium and can co-purify as a contaminant along with recombinant Cys-tagged polypeptides. This is because SlyD has a polycysteine region that binds the biarsenical-containing separation medium, causing it to co-purify as a contaminant with recombinant Cys-tagged polypeptides.

SlyD also has a polyhistidine region and has been reported to copurify as a contaminant in nickel IMAC purification of 6×His-tagged polypeptides, presumably by interacting with the nickel-containing separation medium used in such IMAC procedures. In recognition of this, others have used E. coli cells lacking SlyD for nickel IMAC purification of desired 6×His-tagged polypeptides (Roof et al., J. Biol. Chem. 269:2902-2910, 1994, available on-line at http://wwwjbc.org/cgi/reprint/269/4/2902; Wülfing et al., J. Biol. Chem. 269:2895-2901, 1994; and Scholz et al., FEBS Lett. 1999 Jan 29;443(3):367-369, 1999).

SlyD also can interact with biarsenical detection reagents. Biarsenical detection bind to recombinant Cys-tagged polypeptides to yield labeled recombinant polypeptides that can be detected by virtue of a detectable moiety of the detection reagent. Problematically, biarsenical detection reagents also can bind to SlyD, via its polycysteine region (see the tetracysteine motif of SEQ ID NO.:2, and the hexacysteine motif of SEQ ID NO.:3). Such labeling of SlyD can create background that interferes with the detection of labeled recombinant Cys-tagged polypeptides.

In view of the above findings, the invention provides compositions and methods that can reduce or eliminate purification and detection difficulties caused by SlyD. In some embodiments, the compositions and methods of the invention involve a host cell that has been engineered to eliminate or reduce the amount of SlyD. Such a host cell lacks or contains a reduced amount of SlyD, relative to the cell from which it was derived. In other embodiments, the compositions and methods of the invention involve a host cell engineered to contain a SlyD polypeptide that has been mutated to reduce or eliminate its ability to bind biarsenical reagents.

In other embodiments, the compositions and methods of the invention involve anti-SlyD antibodies that can be used to remove SlyD from a mixture comprising desired biomolecule(s). In such embodiments, types of molecules other than antibodies may be used, so long as they bind slyD. Preferably, the anti-SlyD antibody or SlyD-binding molecule binds specifically to SlyD.

As used herein, the term SlyD refers to the E. coli SlyD polypeptide of SEQ ID NO.: 1 and homologous polypeptides that can bind biarsenical reagents. SlyD polypeptides in other organisms can be identified by homologous nucleotide and polypeptide sequence analyses. For example, performing a query on a database of nucleotide or polypeptide sequences can identify SlyD homologs. Homologous sequence analysis can involve BLAST or PSI-BLAST analysis of non-redundant databases. Polypeptides in the database that have greater than 40% sequence identity to SEQ ID NO. 1 are candidates for evaluating their ability to bind biarsenical reagents. If desired, manual inspection of such candidates can be carried out to narrow the number of candidates for evaluation. Manual inspection is performed by selecting those candidates that appear to have polycysteine motifs.

A percent identity for a “subject” nucleic acid or amino acid sequence relative to a “target” nucleic acid or amino acid sequence can be determined as follows. First, a target nucleic acid or amino acid sequence of the invention can be compared and aligned to a subject nucleic acid or amino acid sequence, using the BLAST 2 Sequences (B12seq) program from the stand-alone version of BLASTZ containing BLASTN and BLASTP (e.g., version 2.0.14). The stand-alone version of BLASTZ can be obtained at <www.fr.com/blast> or <www.ncbi.nlm.nih.gov>. Instructions explaining how to use BLASTZ, and specifically the B12seq program, can be found in the ‘readme’ file accompanying BLASTZ. The programs also are described in detail by Karlin et al, 1990, Proc. Natl. Acad. Sci. 87:2264; Karlin et al, 1993, Proc. Natl. Acad. Sci. 90:5873; and Altschul et al, 1997, Nucl. Acids Res. 25:3389. B12seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per residue cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acid residues (i.e., excluding gaps) from the target sequence that align with sequence from the subject sequence starting with any matched position and ending with any other matched position. A matched position is any position where an identical nucleotide or amino acid residue is present in both the target and subject sequence. Gaps of one or more residues can be inserted into a target or subject sequence to maximize sequence alignments between structurally conserved domains (e.g., alpha-helices, beta-sheets, and loops). The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the B12seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90). The amino acid sequence of a SlyD homolog has 40% or greater (e.g., >90%, >80%, >70%, >60%, or >50%) sequence identity to SEQ ID NO. 1. A nucleic acid or amino acid target sequence that aligns with a subject sequence can result in many different lengths with each length having its own percent identity. The length value will always be an integer. The percent identity value is can be rounded to the nearest tenth (e.g., 78.11, 78.12, 78.13 and 78.14 are rounded down to 78.1, while 78.15, 78.16, 78.17, 78.18 and 78.19 are rounded up to 78.2).

III. Host Cells

The invention provides host cells (e.g., bacterial, yeast, mammalian, insect, or plant cells). Nucleic acids encoding desired recombinant polypeptides can be introduced into host cells in accord with the invention. In addition, lysates and extracts of host cells in accord with the invention can be used to make in vitro transcription/translation (IVTT) systems to which nucleic acids encoding desired recombinant polypeptides can be added. Preferred host cells in accordance with the invention have been engineered or manipulated so as to: 1) eliminate or reduce the amount of SlyD; and/or 2) contain a SlyD polypeptide mutated to reduce or eliminate its ability to bind biarsenical reagents. By way of non-limiting example, host cells can be manipulated so as to have less SlyD by treatment with an antisense or RNAi nucleic acid having sequences complementary to or derived from a slyD nucleotide sequence. Non-limiting examples of host cell engineering include the introduction of a mutation into, or the deletion of, an endogenous slyD gene; and the over-expression of a mutant slyD gene that has been introduced. into a host cell by means of an expression vector

Suitable bacterial hosts include gram negative and gram positive bacteria of any genus that include SlyD, including Escherichia sp. (e.g., E. coli), Klebsiella sp., Streptomyces sp., Streptocococcus sp., Shigella sp., Staphylococcus sp., Erwinia sp., Klebsiella sp., Bacillus sp. (e.g., B. cereus, B. subtilis and B. megaterium), Serratia sp., Pseudomonas sp. (e.g., P. aeruginosa and P. syringae) and Salmonella sp. (e.g., S. typhi and S. typhimurium). Bacterial strains and serotypes suitable for the invention can include E. coli serotypes K, B, C, and W. A typical bacterial host is E. coli strain K-12. Host cells in accord with the invention are isolated (i.e., separated at least partially from other organisms and materials with which they are associated in nature).

Host cells that lack or contain a reduced amount of SlyD can be engineered by, e.g., mutating a gene encoding SlyD to ehminate it, prevent its expression (i.e., transcription and or translation), or destabilize the transcript or encoded polypeptide; by mutating cis-acting genetic regulatory elements or trans-acting regulatory factors that affect SlyD expression; or by mutating genetic regulatory elements that regulate the expression of trans-acting regulatory factors that affect SlyD expression. Antisense RNA molecules (e.g., targeted to transcripts of a gene encoding SlyD, or transcripts for trans-acting regulatory factors that affect SlyD expression also can be used to eliminate or reduce the amount of SlyD in a host cell.

Such genetic manipulation and engineering techniques can be practiced as a matter of routine experimentation by those skilled in the art.

Host cells that contain a mutant SlyD that exhibits reduced or eliminated ability to bind biarsenical reagents also can be made by routine experimentation (e.g., by site-directed mutagenesis in combination with a recombination technique). Suitable mutations can delete one or more amino acids (e.g., cysteine amino acids in its polycysteine region). Other suitable mutations can substitute one or more amino acids (e.g., cysteine amino acids in its polycysteine motif).

Several exemplary host strains have been deposited in the Agricultural Research Service Patent Culture Collection maintained by the National Center for Agricultural Utilization Research in Peoria, Ill., USA (see Table 1 for genotypes and accession numbers). Each of the deposited strains have been engineered to lack the SlyD polypeptide of SEQ ID NO. 1. Other utant strains are known in the art (Table 1).

TABLE 1
SLYD STRAINS

| | | Accession No. |
|---|---|---|
| Strain | Genotype | or Publication |
| JDP670 | F⁻ ompT hsdS_{B} (r_{B}⁻m_{B}⁻) gal dcm slyD::kan | B-30688 |
| | (DE3) | |
| JDP671 | F⁻ araD139 delta(argF-lac)U169 prsL150 | B-30689 |
| | relA1 deoC1 rbsR fthD5301 fruA25 slyD1 | |
| | Tn10 (Tet⁻R) lambda- | |
| JDP687 | Hfr rna-19 gdhA2 his-95 relA1 spoT1 metB1 | B-30690 |
| | slyD1 Tn10 (Tet⁻R) | |
| JDP689 | Hfr rna-19 gdhA2 his-95 relA1 spoT1 metB1 | B-30691 |
| | slyD::kan | |
| JDP694 | F⁻ ompT hsdS_{B} (r_{B}⁻m_{B}⁻) gal dcm slyD1 | B-30692 |
| | (DE3) | |
| JDP704 | F⁻ ompT hsdS_{B} (r_{B}⁻m_{B}⁻) gal dcm rne131 | B-30693 |
| | slyD1 (DE3) | |
| JDP707 | F⁻ ompT hsdS_{B} (r_{B}⁻m_{B}⁻) gal dcm rne131 | B-30694 |
| | slyD::kan (DE3) | |
| | RY7425 (CQ21 zac::Tn10kan zhd::Tn10) | PNAS 97: 4297 |
| | slyD1 | (2000) |
| | A19 slyD::kan | * |

* U.S. Provisional patent application No. 60/587,583, filed Jul. 14, 2004.

Bacterial hosts of the invention include those disclosed herein, as well as derivatives and/or progeny host cells thereof. A “derivative” bacterium is described with reference to a specified “parent” or “ancestor” bacterium. A derivative bacterium can be made by introducing one or more mutations (e.g., addition, insertion, deletion or substitution of one or more nucleic acids) in the chromosome of a specified bacterium (e.g., a parent or ancestor bacterium). For example, one or more of the E. coli K-12 nucleic acid open reading frames identified in RefSeq: NC_{—}000913 (derived from GenBank: U00096, both of which are incorporated by reference) can be subjected to mutagenesis. A derivative bacterium also can be made by introducing one or more mutations (e.g., addition, insertion, deletion or substitution of one or more nucleic acids) in an extrachromosomal nucleic acid present in a specified bacterium. A derivative bacterium can be made by adding one or more extrachromosomal nucleic acids (e.g., plasmid or F′ episome) to a specified bacterium. A derivative bacterium also can be made by removing (e.g., by “curing”) extrachromosomal nucleic acids from a specified bacterium. Techniques for making all such derivatives can be practiced as a matter of routine by those of skill in the art.

IV. In Vitro Protein Synthesis (IVPS) Systems

Examples of such systems and other related embodiments are disclosed in U.S. Provisional Patent Application No. ______, filed Oct. 1, 2004, “Feeding Buffers, Systems, and Methods for In Vitro Synthesis,” naming as inventors, Wieslaw Antoni Kudlicki, Sharanthi Keppetipola, Julia Feltcher, Ashley Getbehead, Frederico Katzen and Laura Vozz-Brown (attorney docket No. 0942.6660000), U.S. patent application Ser. No. 10/091,538, filed Mar. 7, 2002, and U.S. Provisional Patent Application No. 60/587,583, filed Jul. 14, 2004, the disclosures of which applications are incorporated by reference herein in their entireties.

Cell extracts have been developed that support the synthesis of proteins in vitro from purified mRNA transcripts, or from mRNA transcribed from DNA during the in vitro synthesis reaction. Such protein synthethis systems are called “IVPS systems” herein, IVPS being an acronym for “In Vitro Protein Synthesis.”

Both prokaryotic cells and eukaryotic cells can be used for protein and/or nucleic acid synthesis according to the invention (see, e.g., Pelham et al, European Journal of Biochemistry, 67: 247, 1976).

Prokaryotic systems benefit from simultaneous or “coupled” transcription and translation. Eukaryotic IVPS systems include without limitation rabbit reticulocyte lysates, and wheat germ lysates.

To date, several systems have become available for the study of protein synthesis and RNA structure and function. To synthesize a protein under investigation, a translation extract must be “programmed” with an mRNA corresponding to the gene and protein under investigation. The mRNA can be produced from DNA, or the mRNA can be added exogenously in purified form. Historically, such mRNA templates were purified from natural sources or, using more recently developed technologies, prepared synthetically from cloned DNA using bacteriophage RNA polymerases in an in vitro reaction.

More recently, techniques using coupled or complementary transcription and translation systems which carry out the synthesis of both RNA and protein in the same reaction have been developed (IVTT). The cell extracts used for the modem techniques must contain all the components necessary both for transcription (to produce mRNA) and for translation (to synthesize protein) in a single system. In such a system, the input template is DNA, which is normally much easier to obtain than RNA and much more readily manipulable.

An early coupled system was based on a bacterial extract (Lederman and Zubay, Biochim. Biophys. Acta, 149: 253, 1967). Since prokaryotes normally carry out a coupled reaction within their cytoplasm, this bacterial based system closely reflected the in vivo process. This general system has seen widespread use for the study of prokaryotic genes.

However, this general bacterial system is generally not useful for eukaryotic genes, due to its inefficiency and relatively high nuclease content. In vitro synthesis circumvents many of these problems (see Kim and Swartz, Biotechnol. Bioeng. 66:180-188, 1999; and Kim and Swartz, Biotechnol. Prog. 16:385-390, 2000). Also, through simultaneous and rapid expression of various proteins in a multiplexed configuration, this technology can provide a valuable tool for development of combinatorial arrays for research, and for screening of proteins. In addition, various kinds of unnatural amino acids can be efficiently incorporated into proteins for specific purposes (Noren et al., Science 244:182-188, 1989).

In some embodiments, the invention relates to, or uses as an assay, Invitrogen's Expressway™ and Tag-On-Demand™ IVPS systems. These include without limitation Expressway™ systems described in detail in the following Manufacturer's Instruction Manuals for these products, all of which are incorporated by reference:

Expressway™ In Vitro Protein Synthesis System Manual, Version C, Apr. 11, 2003 (on the worldwide web at http://www.invitrogen.com/content/sfs/manuals/expressway_man.pdf);

Expressway™ Linear Expression System Manual, Version A, 26 Sept. 2003 (see http ://www.invitrogen.com/content/sfs/manuals/expresswaylinear_an.pdf);

Expressway™ Linear Expression System with TOPO® Tools Technology, Version A, 26 Sep. 2003 (see http://www.invitrogen.com/content/sfs/manuals/expresswaylinearwithtopotools_man.pdf);

Expressway Plus Expression System Manual, Version A, 26 Sept. 2003 (see http://www.invitrogen.com/content/sfs/manuals/expresswayplus_man.pdf); and

Expressway Plus Expression System with Lumio Technology Manual, Version B, 27 Feb. 2004 (see http://www.invitrogen.com/content/sfs/manuals/expresswayplus_lumio_man.pdf).

Two components of Invitrogen's E. coli expression systems, the Expressway™ Systems, are a crude cell-free S30 extract and a translation buffer. The S30 extract contains the majority of soluble translational components including initiation, elongation and termination factors, ribosomes and tRNAs from intact cells. The translation buffer contains energy sources such as ATP and GTP, energy regenerating components such as phosphoenol pyruvate/pyruvate kinase, acetyl phosphate/acetate kinase or creatine phosphate/creatine kinase and a variety of other important co-factors (Zubay, Ann. Rev. Genet. 7:267-87, 1973; Pelham and Jackson, Eur J Biochem. 67:247, 1976; and Erickson and Blobel, Methods Enzymol. 96;38-50, 1983).

The Expressway™ Plus Expression System utilizes a coupled transcription and translation reaction to produce active recombinant protein. The Expressway™ Plus System provides all the components for cell-free protein production. The kit includes an E. coli extract containing the cellular machinery required to drive transcription and translation. The IVPS Plus reaction buffer is also included in the kit and contains the required amino acids (except methionine) and an ATP regenerating system for energy. The reaction buffer, methionine, T7 Enzyme Mix, and DNA template of interest, operably linked to a T7 promoter, are mixed with the E. coli extract. As the DNA template is transcribed, the 5′ end of the mRNA is bound by ribosomes and undergoes translation as the 3′ end of the template is still being transcribed.

The Expressway™ Linear Expression System is used for rapid high-yield in vitro expression from linear DNA templates. The system uses an E. coli extract optimized for expression of full-length, active protein from linear templates. As a result, linear templates are more stable during transcription and translation, resulting in higher yields of properly folded products. In the Expressway™ Linear Expression System, at least two options are available for generating T7 promoter-driven templates. The Expressway™ Linear Expression Kit can be used to express PCR templates generated from a plasmid containing the appropriate elements for expression (T7 promoter, ribosome binding site, T7 termination sequence). The Expressway™ Linear Expression Kit with TOPO® Tools includes a 5′ and 3′ element that can be operably joined to a PCR product. The 5′ element contains a T7 promoter, ribosome binding site, and start codon. The 3′ element contains a V5 epitope tag followed by a 6×His region and a T7 terminator. The TOPO® Tools elements are joined to the PCR product in a TOPO® ligation reaction and then amplified by PCR.

The Expressway™ Plus Expression System with Lumio™ Technology kit includes IVPS Lumio™ E. coli Extract, IVPS Plus E. coli Reaction Buffer, RNase A, T7 Enzyme Mix, Methionine, reaction tubes, pEXP3-DEST vector, a control plasmid, and a Lumio™ Green Detection Kit or components thereof. See Keppetipola et al., Rapid Detection of in vitro expressed proteins using Lumio™ Technology. Focus 25.3:7, 2003.

In addition to prokaryotic system extracts, eukaryotic system extracts have also been developed. These eukaryotic systems use exogenously added E.coli RNA polymerase or wheat germ RNA polymerase to transcribe exogenous DNA. These systems have had limited success for the general study of eukaryotic genes, due to their low efficiency, and to the fact that they were developed and used prior to the widespread success of cDNA cloning techniques. Other coupled systems have been developed for the study of viral protein synthesis, but are not generally useful for non-viral templates.

In the mid-1980s, the development of more efficient in vitro transcription systems, particularly ones using phage polymerases such as T7; SP6 and T3, allowed protein synthesis systems to be defined that more efficiently translated cloned mRNA sequences in vitro using translation extracts from wheat germ and rabbit reticulocytes. For example, Perara and Lingappa (J. Cell Biol., 101: 2292-2301 (1985)) showed that SP6 RNA polymerase transcription reactions could be added directly to reticulocyte lysate for the production of protein.

This insight illuminated the need to purify the mRNA prior to translation. Later other workers showed that the transcription and translation could be coupled in reticulocyte lysate by including a phage polymerase and appropriate transcriptional co-factors in the reaction (Spirin et al, Science, 242: 1162-1164 (1988); Craig et al, Nucleic Acids Res., 20: 4987-4995 (1992)). More recently, U.S. Pat. No. 5,324,637 to Thompson et al described a coupled transcription and translation system in eukaryotes. Thompson used reticulocyte lysate and a phage polymerase where the coupling of the two reactions was facilitated by specific conditions, notably the concentration of magnesium ions, which permitted both transcription and translation to occur in the same reaction mix.

Although the coupled approach for transcription and translation systems is useful for many proteins, translation efficiencies vary widely depending on the type of DNA template which is used (e.g., supercoiled plasmid DNA or linear DNA). In addition, the amount of mRNA synthesized in a coupled reaction is difficult to control under most coupled conditions, such as disclosed in Thompson. Since efficiency and fidelity of translation are dependent upon the amount of mRNA added to and present during the reaction, a possible explanation for the undesirable variability of results obtained using these coupled systems, in which the reactions occur simultaneously, is that transcription and translation are not consistent between various templates under the conventional reaction conditions.

A number of subsequent improvements have been made (see e.g., Kim et al, Eur. J. Biochem., 239: 881-886, (1996); Patnaik and Swartz, Biotechniques, 24: 862-868, (1998); and Kim and Swartz, Biotech. and Bioeng., 66: 180-188, (1999)) to improve the IVTT system. One of the main problems, however, of the conventional IVTT systems is that these systems do not produce sufficient quantities of protein for extensive analysis of protein(s) of interest.

The conventional inefficient protein synthesis can be in part attributed to factors such as maintenance of an energy supply, the stability of the DNA template for transcription and the stability of mRNA for translation.

The problem of DNA template stability is especially evident when linear substrates, such as PCR derived products or restriction enzyme(s) digested fragments, are used in cell-free extracts for generating protein(s). The linear DNA fragments are susceptible to rapid degradation by intracellular exonucleases of E.coli, particularly RecBCD (Pratt et al, Nucleic Acids Res., 9: 4459-4474, (1981); Benzinger et al, J. Virol., 15: 861-871, (1975); Lorenz and Wackemagel, Microbiol Rev., 58, 563-602, (1994)) and possibly by other nucleases.

In most cases, a supercoiled plasmid DNA containing the gene of interest is used in IVTT systems because plasmid DNAs are more stable (Kudlicki et al, Anal. Biochem., 206: 389-393, (1992)). Linear DNAs are more readably degraded by DNA nucleases, especially DNA exonucleases, such as RecBCD. Mutant RecBCD strains devoid of the exonuclease have been made. These mutant strains do not so rapidly degrade linear DNA; however, such mutant strains grow extremely poorly and therefore do not produce satisfactory results (Yu et al, PNAS, 97: 5978-5983, (2000)).

E.coli extract for cell-free protein synthesis has been made using a RecD mutant of E.coli (Lesley et al, J. Biol. Chem., 266: 2632-2639, (1991)). However, cell-free extract made using RecD mutant E.coli contained high level of chromosomal DNA contamination because sheared chromosomal DNA is not degraded by the nuclease that has been mutated. To remedy this, micrococcal nuclease has been added to degrade the contaminating chromosomal DNA to minimize background. Similarly, entire RNase E deletion mutants have been made, but cell growth of these complete deletion mutants is also poor and unsuitable for providing a cell free extract. In addition, the cell-free extract made from such mutants did not enhance protein production and for unknown reason, the protein synthesis is independent of T7 RNA polymerase addition even though the PCR product contained T7 promoter. The present invention provides a cellular extract that includes an extract from an organism whose genome in wild type organisms includes a SlyD gene, wherein the extract is substantially free of a SlyD polypeptide that binds a bi-arsenical reagent. In certain aspects, the cellular extract or a buffer mixed with the extract, additionally includes at least one other component of any of the components in Chaterjee et al., U.S. Pub. Pat. App. No. 2002/0168706, incorporated herein in its entirety. For example, the cellular extract can include one inhibitor of at least one enzyme, e.g., an enzyme selected from the group consisting of a nuclease, a phosphatase and a polymerase; and optionally the extract can be modified from a native or wild type extract to exhibit reduced activity of at least one enzyme, e.g., an enzyme selected from the group consisting of a nuclease, a phosphatase and a polymerase; and at least two energy sources that supply energy for protein and/or nucleic acid synthesis. In certain aspects the extract includes the Gam protein.

V. Puririfcation and Detection

The invention also provides methods for purifyng and detecting desired biomolecules (e.g., recombinant polypeptides).

Recombinant polypeptides typically are produced using a host cell (or derivative thereof) into which a nucleic acid that can give rise to the desired polypeptide has been introduced. Such a nucleic acid may continue to exist as an extrachromosomal element or may integrate into the host cell genome. Methods for producing recombinant polypeptides in host cells can be practiced as a matter of routine experimentation by those skilled in the art. For example, routine protocols for rendering bacteria capable of taking up and maintaining exogenous polynucleotides (i.e., making them “transformable” or “competent”), and for transforming them are well known to the skilled practitioner.

Recombinant polypeptides also can be made using an IVTT system. An IVTT system contains all of the biomolecules required for transcription and translation. Methods for making IVTT systems and for producing recombinant polypeptides in IVTT systems can be practiced as a matter of routine experimentation by those skilled in the art. Such methods typically involve adding a nucleic acid that can give rise to a recombinant polypeptide to a cell lysate or extract that can support transcription and translation. Recombinant polypeptides made using an IVTT system can themselves be subjected to purification and detection.

Recombinant polypeptides can include one or more detection and/or purification tags. Purification and detection tags are well known in the art and include peptides such as polyhistidine motifs, polycysteine motifs, streptavidin, biotin, antigenic epitopes, glutathione-S-transferase, beta-galactosidase, and beta-amylase. Nucleic acids that encode a purification tag can be combined with a nucleic acid encoding a desired polypeptide to make a nucleic acid that encodes a tagged recombinant polypeptide. In some cases the resultant nucleic acid “expression construct” can give rise to the tagged recombinant polypeptide, e.g., after it is introduced into a host cell or added to a host cell extract.

Polycysteine tags (Cys-tags) can interact with biarsenical reagents, and are one type of detection/purification tag (see, e.g., U.S. Pat. Nos. 6,054,271; 6,008,378; 5,932,474; 6,451,569; WO 99/21013; U.S. Provisional Patent Application No. 60/513,031, filed Oct. 22, 2003; which are incorporated into the present disclosure by reference). A Cys-tag can vary in size and typically contains at least 6 (e.g., 5-10, 10-15, or 15-20) amino acids. A Cys-tag can be present at the N-terminus, C-terminus, and/or internal to a recombinant polypeptide. In general, a Cys-tag includes two or more cysteines that are in an appropriate configuration for interacting with the biarsenical molecule. Cys-tags typically are alpha-helical and include at two to ten (e.g., 2, 3, 4, 5 or 6) cysteine amino acids. Typically, the Xaa amino acids have a high propensity to form alpha-helical structures. A Cys-tag may be arranged such that the side chains of two pairs of cysteines are exposed one the same face of an alpha-helix. An exemplary Cys-tag is the peptide CCXaaXaaCC, wherein each Xaa is any amino acid. The cysteines in this Cys-tag are positioned to encourage arsenic interaction across helical turns. A Cys-tag need not be completely helical to react with a biarsenical reagent. For example, reaction of a first arsenic of a biarsenical with a pair of cysteines may nucleate an alpha-helix and position two other cysteines favorably for reacting with a biarsenical molecule.

Purifying a desired biomolecule involves separating it (completely or partially) from at least one contaminant. Desired molecules can be purified from undesired contaminants purified via one or more purification steps. Some purification processes can result in a “homogeneous” preparation comprising at least about 70% (e.g., at least about 80%, at least about 90% by weight, or at least about 95%) by weight of the desired biomolecule(s). Other purification processes (e.g., obtaining a cell lysate, cell extract or cell culture supernatant) can result in a lower degree of purification, which may nonetheless be suitable for a particular use. For example, cell lysates and cell extracts can be used to make an IVTT system.

Steps for purifying desired biomolecule(s) from cultured cells can depend on whether the desired biomolecule(s) remains inside cultured cells or are secreted into the cell culture growth medium. For desired biomolecules that remain within cultured cells, purification typically involves disrupting the cells (e.g., by mechanical shear, freeze/thaw, osmotic shock, chemical treatment, and/or enzymatic treatment). Such disruption results in a cell lysate that contains the desired biomolecule and other cellular constituents. In some cases, much of the undesired cellular material can be removed by filtration or centrifugation to yield a cell extract that contains the partially purified biomolecule. For desired biomolecules that are secreted into the cell culture growth medium, undesired ceHular constituents present less of a problem, although host cell constituents can be present in the culture medium (e.g., as a consequence of natural cell death). Secreted biomolecules can be purified by separating the culture medium from all or most of the cultured cells (e.g., by centrifugation or filtration).

Chromatographic techniques often are used to further purify a desired polypeptide from cell culture growth medium, products of cellular metabolism, and/or other cellular constituents. Such techniques can separate polypeptides on the basis of, e.g., size, charge, hydrophobicity, or presence of purification tags. Chromatographic separation schemes can be tailored to particular desired polypeptides, using one or more chromatographic techniques and/or separation media. During chromatographic separation, a desired polypeptide can move at a different rate through a separation medium, or can adhere selectively to the separation medium, relative to undesired molecules. In addition, a desired polypeptide can be positively selected or negatively selected. Thus, in some chromatographic separation schemes, desired molecules can be separated from undesired molecules when the undesired molecules adhere to the separation medium and the desired molecule not (negative selection). In such a scheme, desired molecules are present in the eluate or flow-through and undesired molecules are retained in association with the separation medium. Alternatively, desired molecules can be separated from undesired molecules when desired molecules adhere to the separation medium and undesired molecules do not (positive selection). In such a scheme, the eluate or flow-through contains undesired molecules, and desired molecules are retained in association with the separation medium. The desired molecules can be then be recovered, e.g., by exposing the separation medium to a chemical or enzymatic agent suitable for dissociating the desired polypeptide.

Ion exchange chromatography is one chromatographic technique that can be used to purify desired polypeptides. In ion exchange chromatography, charged portions of molecules in solution are attracted by opposite charges of an ion exchange medium (e.g., contained in an ion exchange chromatography column), when the ionic strength of the solution is sufficiently low. Solutes can be dissociated from an ion exchange medium and eluted from an ion exchange column by increasing the ionic strength of the solution. Changing the pH to alter solute charge is another way to dissociate solutes from an ion exchange medium. Ionic strength and/or pH can be changed gradually (gradient elution) or stepwise (stepwise elution).

Metal ion affinity chromatography (MIAC) is another chromatographic technique that can be used to purify desired molecules (e.g., recombinant polypeptides). MIAC is an affinity chromatography technique that involves the binding of desired molecules to metal ions.. Immobilized metal ion affinity chromatography (IMAC) is a MIAC technique that involves the use of a separation medium to which metal ions have been chelated. Desired polypeptides can be immobilized on such a metal chelate substrate, reportedly via interaction(s) between metal ion(s) and electron-donating amino acid(s) such as histidine and cysteine. Thus, IMAC routinely is used to purify recombinant polypeptides that include polyhistidine or polycysteine motifs (tags). Whether, and with what affinity, a particular desired polypeptide will bind to a metal chelate substrate can depend on the conformation of the polypeptide, the number of available coordination sites on the chelated metal ion ligand, and the number of amino acid side chains available to bind the chelated metal ion ligand.

Electrophoresis techniques also are used to purify desired polypeptides. Electrophoresis is based on the principle that charged particles migrate in an applied electrical field. If electrophoresis is carried out in solution, molecules are separated according to their surface net charge density. If carried out in semisolid materials (gels), the matrix of the gel adds a sieving effect so that particles migrate according to both charge and size.

Gel-based electrophoresis can be carried out in a variety of formats, including in standard-sized gels, minigels, strips, gels designed for use with microtiter plates and other high throughput (HTS) applications, and the like. Two commonly used media for gel electrophoresis and other separation techniques are agarose and polyacrylamide. In general, electrophoresis gels can be either in a slab gel or tube gel form.

Electrophoresis can performed in the presence of a charged detergent like sodium dodecyl sulfate (SDS) which coats, and thus equalizes the charges of, most polypeptides, so that migration depends on size (molecular weight). Polypeptides often are separated in this fashion, i.e., SDS-PAGE (PAGE=polyacrylamide gel electrophoresis). In addition to SDS, one or more other denaturing agents, such as urea, can be used to minimize the effects of secondary and tertiary structure on the electrophoretic mobility of polypeptides. Such additives typically are not necessary for nucleic acids, which have a similar surface charge irrespective of their size and whose secondary structures generally are broken up by the heating of the gel that happens during electrophoresis.

Isoelectric focusing (IEF) is an electrophoresis technique that involves passing a mixture through a separation medium having a pH gradient or other pH function. An IEF system has an anode at a position of relatively low pH end and a cathode disposed at another position of higher pH. Molecules having a net positive charge under the acidic conditions near the anode will move away from the anode. As they move through the IEF system, molecules enter zones having less acidity, and their positive charges decrease. Each molecule will stop moving when it reaches a point in the system having a pH equivalent to its isoelectric point (pI). This effectively separates molecules that have different pI values.

Two-dimensional (2D) electrophoresis involves a first electrophoretic separation in a first dimension, followed by a second electrophoretic separation in a second, transverse dimension. In a common 2D electrophoretic method, polypeptides are subjected to IEF in a polyacrylamide gel in the first dimension, resulting in separation on the basis of pI, and are then subjected to SDS-PAGE in the second dimension, resulting in further separation on the basis of size.

Capillary electrophoresis (CE) achieves molecular separations on the same basis as conventional electrophoretic methods, but does so within the environment of a narrow capillary tube (25 to 50 μm). The main advantages of CE are that very small (e.g., nanoliter) volumes of sample are required and that separation can be performed very rapidly, thus increasing. sample throughput relative to other electrophoresis formats. Examples of CE include capillary electrophoresis isoelectric focusing (CE-IEF) and capillary zone electrophoresis (CZE). Capillary zone electrophoresis (CZE) is a technique that separates molecules on the basis of differences in mass to charge ratios, which permits rapid and efficient separations of charged substances. In general, CZE involves introducing a sample into a capillary tube and applying an electric field to the tube. The electric potential of the field pulls the sample through the tube and separates it into its constituent parts. Constituents of the sample having greater mobility travel through the capillary tube faster than those with slower mobility. As a result, the constituents of the sample are resolved into discrete zones in the capillary tube during their migration through the tube. An on-line detector can be used to continuously monitor the separation and provide data as to the various constituents based upon the discrete zones.

Electrophoretic purification and chromatographic purification can be performed for analytic purposes (e.g., where the objective is to detect the presence or absence of a desired molecule) or for preparative purposes (e.g., where the objective is to recover a desired molecule for further treatment, analysis or use).

Purified biomolecules can be detected using any known detection technique or reagent. One way to detect recombinant polypeptides involves the use of detection reagents that bind to detection tags. Such detection reagents include a detectable moiety. A detectable moiety can be detected directly, indirectly by virtue its interaction with another directly detectable molecule, indirectly by interacting with another molecule to produce a directly detectable molecule. For example, a detectably labeled antibody that can be used to detect recombinant polypeptides having cognate antigenic epitopes. As another example, a biarsenical detection moiety can be used to detect Cys-tagged recombinant polypeptides.

VI. FLASH/Lumio

The invention is drawn to compositions, methods and kits for labeling tetracysteine-tagged proteins with arsenical molecules, preferably biarsenical fluorophores, with increased specificity, including compositions, methods and kits particularly adapted for labeling of tetracysteine-tagged proteins to be resolved within an electrophoresis gel.

The Fluorescein Arsenical Hairpin binding (FlAsH™) labeling reagent, EDT2[4′,5′-bis(1,3,2-dithioarsolan-2-yl)fluorescein-(1,2-ethanedithiol)2], is a bisarsenical compound that binds to polypeptides comprising the sequence, C-C-X-X-C-C, wherein “C” represents cysteine and “X” represents any amino acid other than cysteine (Griffin et al. Science 281:269-272, 1998). Adams et al. (Am Chem Soc. 124:6063-6076, 2002) have reported that the highest affinity is achieved when X-X is proline and glycine. FlAsH tags have been successfully incorporated at either the N- or C-termini of proteins, as well as exposed surface regions within a protein (Griffin et al., 1998; Adams et al., 2002; and Griffin et al. Methods Enzymol. 327:565-78, 2000).

The bisarsenical dye is normally reacted with two ethylenedithiol (EDT) molecules for easier diffusion through the cell membrane. The FLASH™-EDT2 labeling reagent is non-fluorescent and becomes fluorescent upon binding to the “FLASH-tag” tetracysteine motif. When the FlAsH-EDT2 dye is not bound to a protein, the small size of the EDT permits the free rotation of the arsenium atoms that quench the fluorescence of the fluorescein moiety. When a C-C-P-G-C-C labeled protein is mixed with the FlAsH-EDT2 dye, the arsenium atoms of the FlAsH™ dye react with the tetracysteine tag of the protein and form covalent bonds. The product of this reaction does not allow free rotation of the arsenium atoms and, because they no longer quench its fluorescence, the fluorescein moiety becomes fluorescent. The increase of the fluorescence is about 50,000 fold when the FlAsH dye is bound to protein (Griffin et al., 1988).

This quenching of the fluorescence of the FlAsH dye when not bound and recovering the full fluorescence when bound makes it highly suitable for detection of proteins. Although the FlAsH dye can react with other cysteines in the protein molecule that are not part of the FlAsH tag, the affinity for the other cysteines is significantly lower. Therefore; a small amount of a protein containing the FlAsH tag can be detected in the presence of large quantities of other proteins.

The FlAsH-EDT2 reagent is also useful for in cell assays because this reagent can freely diffuse across the cell membranes of live mammalian cells and bind to proteins engineered to contain the FlAsH-tag. This allows for in vivo detection and subcellular localization of specific proteins without the need for time-consuming immunostaining (Griffin et al., 1998; Adams et al., 2002; and Griffin et al., 2000).

In addition to labeling of specific proteins in live cells, the FlAsH-EDT2 reagent can also be used to detect FLASH-tagged proteins in SDS-PAGE gels (Adams et al., 2002). Inclusion of the FlAsH-EDT2 reagent in the sample loading buffer allows rapid detection of recombinant proteins in whole cell lysates using a standard ultraviolet (UV) lightbox, without the need for western blotting or other more laborious protein detection methods.

The FlAsH-EDT2 reagent can also in affinity purification of proteins comprising the C-C-X-X-C-C sequence. Thorn et al. (A novel method of affinity-purifying proteins using a bis-arsenical fluorescein. Protein Sci. 9:213, 2000) report that kinesin tagged with this sequence binds specifically to FlAsH resin and can be eluted in a fully active form. Thorn et al. reported that the protein obtained with a single FlAsH chromatographic step from crude Escherichia coli lysates is purer than that obtained with nickel affinity chromatography of 6×His tagged kinesin. Further, protein bound to the FlAsH column can be completely eluted by dithiothreitol, which is unlike nickel affinity chromatography, which requires high concentrations of imidazole or pH changes for elution.

ReAsH is a variant of FlAsH that is useful for electron microscopy (EM), because it can generate singlet oxygen upon illumination. Singlet oxygen drives localized polymerization of the substrate diaminobenzidene (DAB) into an insoluble form that can be viewed by EM. Because the fluorescent label binds directly to the protein of interest, and the DAB polymer deposits directly nearby the fluorophore, the resolution is better than traditional methods, such as immunogold labeling. Additionally, this technique does not require the diffusion of large antibodies into the fixed specimens. See, for example, Daniel and Postma, Molecular Interventions 2:132, 2002; Gaietta et al., Multicolor and electron microscopic imaging of connexin trafficking. Science 296:503, 2002.

A large family of patents are drawn to FlAsH. These include without limiation, U.S. Pat. No. 5,932,474 to Tsien et al., entitled “Target Sequences for Synthetic Molecules”; U.S. Pat. No. 6,054,271 to Tsien et al., entitled “Methods of Using Synthetic Molecules and Target Sequences”; U.S. Pat. Nos. 6,451,569.and 6,008,378, published U.S. Patent Application 2003/0083373, published PCT Patent Application WO 99/2101.3, all to Tsien et al. and all entitled “Synthetic Molecules that Specifically React with Target Sequences”.

Tetracysteine biarsenical affinity tags (FlAsH™ tags) have been successfully incorporated at either the N- or C-termini of proteins, as well as exposed surface regions within a protein and have been used to permit visualization of recombinant proteins expressed within living cells, and in SDS-PAGE gels (Griffin et al. 1998, Griffin et al. 2000, Adams et al. 2002, supra).

In PAGE gels, inclusion of the FlAsH-EDT2 reagent in the sample loading buffer allows rapid detection of recombinant proteins in whole cell lysates using a standard UV light box without the need for western blotting or other more laborious protein detection methods.

Although the preferred tetracysteine motif occurs rarely in natural proteins, permitting specific labeling of proteins to which the tetracysteine motif has been recombinantly fused, FlAsH-EDT2 has been shown additionally to bind to endogenous cysteine-containing proteins, Stroffekova et al., Pflugers Arch.—Eur. J. Physiol. 442:859-866 (2001), which increases background fluorescence. Stroffekova et al. suggest that FlAsH™ binding to the vicinal cysteines in the C-X-X-C protein motif of endogenous proteins may limit the use of FlAsH-EDT2 to staining recombinant proteins expressed at a high level in cells with a naturally low background.

In the methods of the present invention, the biarsenical fluorophore can usefully be a biarsenical derivative of a known fluorophore, such as fluorescein, usefully FlAsH-EDT2 (Lumio™ Green, Invitrogen Corp., Carlsbad, Calif.), or such as resorufin, usefully ReAsh-EDT2 (Lumio™ Red, Invitrogen Corp., Carlsbad, Calif.), or may instead be an oxidized derivative, such as ChoXAsH-EDT2 or HoXAsH-EDT2.

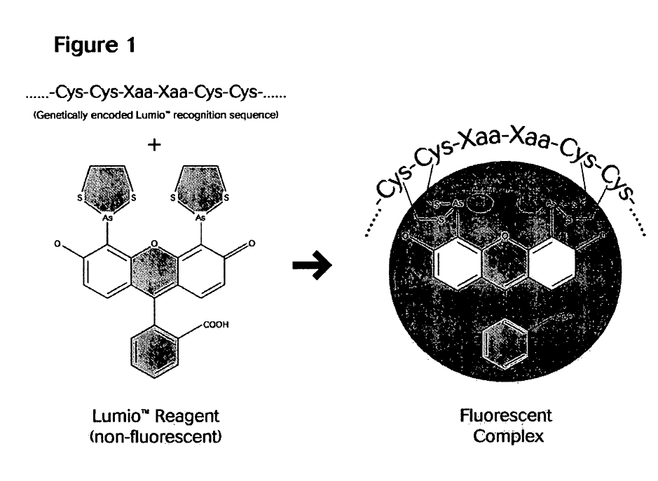

Lumio™ Technology is based on FlAsH, a biarsenical derivative of fluorescein that binds to an engineered tetracysteine sequence (FIG. 1). Lumio™ Technology coupled with a modified Expressway™ Plus System can be used to rapidly and easily detect in vitro expressed proteins. The versatility of the Lumio™ Technology allows co-translational monitoring of protein production during an Expressway™ Plus in vitro synthesis reaction. The Lumio™ Reagent has the useful characteristic of undergoing a marked transition from a virtually non-fluorescent state to a highly fluorescent state upon binding to a tetracysteine sequence. By taking advantage of this novel characteristic, real-time analysis of protein accumulation can be observed. Using a standard fluorescence plate reader, the real-time incorporation of the Lumio™ sequence into newly synthesized proteins can be monitored directly in the in vitro reaction mixture. Due to the site-specific binding of the Lumio™ Green Detection Reagent, quantitative analysis of protein expression levels is possible.

Lumio™ Green Detection Reagent also can be directly added to protein samples before electrophoresis. This permits the visualization of protein products immediately after electrophoresis with a standard UV light box or a laser gel scanner, without the need for radioactivity. The robust, covalent attachment of the Lumio™ Reagent to the tetracysteine sequence eliminates any requirements for protein gel manipulation, such as the need to fix, stain, destain, or dry. In addition, all safety, waste disposal, and regulatory issues associated with the use of radiolabeled amino acids are abolished.

VII. Anti-Slyd Antibodies

The invention also provides antibodies that selectively bind SlyD. Such antibodies can be used to selectively remove SlyD from a mixture containing a desired polypeptide. Any immunopurification technique can be used to accomplish such selective removal. Immunopurification techniques using anti-SlyD antibodies can be used alone or in combination with other purification and detection techniques. Antibodies suitable for the invention include monoclonal antibodies, polyclonal antibodies, multi-specific antibodies, and antibody fragments (e.g., Fab, Fab′, F(ab′)₂, and Fv fragments; diabodies; linear antibodies; single-chain antibodies) that exhibit the desired biological activity and/or binding specificity. Techniques for making and using antibodies and antibody fragments are routine can be practiced as a matter of routine by those skilled in the art.

As is explained in more detail herein, the term “antibody” includes polyclonal, monospecific, monoclonal, camelized, humanized and single-chain antibodies; Fab, Fab′ (Fab′)2 fragments; CDRs; and the like.

Antibodies, Including Monoclonal Antibodies: The term “antibody” is meant to encompass an immunoglobulin molecule obtained by in vitro or in vivo generation of an immunogenic response, and includes both polyclonal, monospecific and monoclonal antibodies. An “immunogenic response” is one that results in the production of antibodies directed to one or more proteins after the appropriate cells have been contacted with such proteins, or polypeptide derivatives thereof, in a manner such that one or more portions of the protein function as epitopes. An epitope is a single antigenic determinant in a molecule. In proteins, particularly denatured proteins, an epitope is typically defined and represented by a contiguous amino acid sequence. However, in the case of nondenatured proteins, epitopes also include structures, such as active sites, that are formed by the three-dimensional folding of a protein in a manner such that amino acids from separate portions of the amino acid sequence of the protein are brought into close physical contact with each other.

Wildtype antibodies have four polypeptide chains, two identical heavy chains and two identical light chains. Both types of polypeptide chains have constant regions, which do not vary or vary minimally among antibodies of the same class (i.e, IgA, IgM, etc.), and variable regions. As is explained below, variable regions are unique to a particular antibody and comprise a recognition element for an epitope.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wildtype antibody are directed to the same epitope of an immunogenic protein, and a single wildtype antibody is thus capable of binding two molecules of the immunogenic protein at the same time.

Types of Antibodies:

Compositions of antibodies have, depending on the manner in which they are prepared, different types of antibodies. Types of antibodies of particular interest include polyclonal, monospecific and monoclonal antibodies.

Polyclonal antibodies are generated in an immunogenic response to a protein having many epitopes. A composition of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

Monospecific antibodies (a.k.a. antipeptide antibodies) are generated in a humoral response to a short (typically, 5 to 20 amino acids) immunogenic polypeptide that corresponds to a few (preferably one) isolated epitopes of the protein from which it is derived. A plurality of monospecific antibodies includes a variety of different antibodies directed to a specific portion of the protein, i.e, to an amino acid sequence that contains at least one, preferably only one, epitope. Methods for producing monospecific antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A monoclonal antibody is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

Variants and derivatives of antibodies include antibody and T-cell receptor fragments that retain the ability to specifically bind to antigenic determinants. Preferred fragments include Fab fragments (i.e, an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (an Fab molecule having two antigen binding domains, each of which may be directed to a different epitope); a single chain Fab chain comprising a variable region, a.k.a., a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more complementarity determining regions (CDRs) sequences of an antibody combining site. The CDR sequences may be linked together on a scaffold when two or more CDR sequences are present.

The term “antibody” also includes genetically engineered antibodies and/or antibodies produced by recombinant DNA techniques and “humanized” antibodies. Humanized antibodies have been modified, by genetic manipulation and/or in vitro treatment to be more human, in terms of amino acid sequence, glycosylation pattern, etc., in order to reduce the antigenicity of the antibody or antibody fragment in an animal to which the antibody is intended to be administered (Gussow et al., Methods Enz. 203:99-121, 1991).

Methods of Preparing Antibodies and Antibody Variants:

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems. Antibodies and antibody variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (see, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Sacharomyces cerevisieae or Pichia pastoras harboring episomal or chromosomally integrated expression constructs; insect cells and viruses such as Sf 9 cells and baculovirus; and mammalian cells harboring episomal or chromosomally integrated (e.g., retroviral) expression constructs (for a review, see Verma et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

XenoMouse® strains are genetically engineered mice in which the murine IgH and Igk loci have been functionally replaced by their Ig counterparts on yeast artificial YAC transgenes. These human Ig transgenes can carry the majority of the human variable repertoire and can undergo class switching from IgM to IgG isotypes. The immune system of the xenomouse recognizes administered human antigens as foreign and produces a strong humoral response. The use of XenoMouse® in conjunction with well-established hybridoma techniques, results in fully human IgG mAbs with sub-nanomolar affinities for human antigens (see U.S. Pat. No. 5,770,429, entitled “Transgenic non-human animals capable of producing heterologous antibodies”; U.S. Pat. No. 6,162,963, entitled “Generation of Xenogenetic antibodies”; U.S. Pat. No. 6,150,584, entitled “Human antibodies derived from immunized xenomice”; U.S. Pat. No. 6,114,598, entitled “Generation of xenogeneic antibodies”; and U.S. Pat. No. 6,075,181, entitled “Human antibodies derived from immunized xenomice”; for reviews, see Green, Antibody engineering via genetic engineering of the mouse: XenoMouse strains are a vehicle for the facile generation of therapeutic human monoclonal antibodies, J. Immunol. Methods 231:11-23, 1999; Wells, Eek, a XenoMouse: Abgenix, Inc., Chem Biol 2000 August; 7(8):R185-6; and Davis et al., Transgenic mice as a source of fully human antibodies for the treatment of cancer, Cancer Metastasis Rev 1999; 1 8(4):421-5).

Hottenrott et al. (Journal of Biological Chemistry 272: 15697-15701, 1997 have described antibodies raised against the complete SlyD protein that are able to detect the N-terminal fragment of SlyD.

In one embodiment of the invention, anti-SlyD antibodies are used to remove SlyD from a mixture containing a Cys-tagged recombinant polypeptide before purification and/or detection of the Cys-tagged polypeptide using a biarsenical reagent. For example, anti-SlyD antibodies can be used to remove SlyD from a cell extract (e.g., IVTT system) containing a Cys-tagged recombinant polypeptide prior to purification and/or detection of the Cys-tagged polypeptide using a biarsenical reagent. In another embodiment of the invention, anti-SlyD antibodies are used to remove SlyD after a mixture containing a Cys-tagged polypeptide has been purified using a biarsenical reagent.

VIII. Kits

The invention also provides kits that include: a host cell in accordance with the invention, a cell extract made from a host cell in accordance with the invention, and/or an anti-SlyD antibody in accordance with the invention.

A host cell typically is provided in one or more sealed containers (e.g., packet, vial, tube, or microtiter plate), which in some embodiments also can contain cell growth media. In some embodiments, the bacterial host is provided in desicated or lyophilized form. In some embodiments, the bacterial host has been rendered competent for transformation. In some embodiments, a kit includes, in separate containers, sterile bacterial nutritional media, reagents for transfection, one or more buffers, and the like.

In some embodiments, a kit includes one or more nucleic acids (e.g., plasmid and/or polymerase chain reaction primer) in a separate container. In some embodiments a kit includes a nucleic acid having an oligonucleotide sequence that encodes a polycysteine motif that can bind a biarsenical reagent. Such a nucleic acid may encode a recombinant Cys-tagged polypeptide, or can be combined with a nucleic acid that encodes a desired polypeptide to make a nucleic acid encoding a desired recombinant Cys-tagged polypeptide.

In some embodiments, a kit includes one or more RNA Polymerases. Non-limiting examples of RNA Polymerases include RNA polymerase II, SP6 RNA polymerase, T3 RNA polymerase, T7 RNA polymerase, and RNA polymerase III. When RNA is to be synthesized from a DNA template, a polymerase active on the DNA molecule of interest should be used. RNA polymerases and transcription factors useful in the invention are well known in the art and will be readily recognized by those skilled in the art.

In some embodiments, a kit includes one or more enzymes useful in gene cloning and expression in a separate container. Non-limiting examples of an enzyme useful in gene cloning and expression include a restriction endonuclease, a nucleic acid polymerase, a nucleic acid ligase, a nucleic acid topoisomerase, a uracil DNA glycosylase, a protease, a phosphatase, ribonuclease, and/or a ribonuclease inhibitor.

A kit typically includes literature describing the properties of the bacterial host (e.g., its genotype) and/or instructions regarding its use for purifying and/or detecting biomolecules such as Cys-tagged recombinant polypeptides.

A kit or composition comprising an anti-SlyD antibody and/or another molecule that specifically binds SlyD can be used in a method of purifying a protein of interest. In this aspect of the invention, the protein of interest can be a His-tagged or a polycysteine protein, and the method of purification can be nickel- or biarsenical-based affinity chromatgography, respectively.

A kit of the invention may further comprise a transfection agent. Non-limitinmg examples of transfection agents are given in Table 2.

TABLE 2
TRANSFECTION AGENTS

| | | PATENTS | |
|---|---|---|---|
| | | AND/OR | AVAILABLE |
| TRANSFECTIONAGENT | DESCRIPTION | CITATIONS | FROM |
| BMOP | N-(2-bromoethyl)- | | |
| | N,N-dimethyl-2,3- | | |
| | bis(9- | | |
| | octadecenyloxy)- | | |
| | propana minimun | | |
| | bromide) | | |
| BMOP:DOPE | 1:1 (wt/wt) | Poult Sci 1997 | |
| | formulation of N-(2- | Jun; 76(6): 882-6. | |
| | bromoethyl)-N,N- | Transfection of | |
| | dimethyl-2,3-bis(9- | avian LMH-2A | |
| | octadecenyloxy)- | hepatoma cells with | |
| | propana minimun | cationic lipids. | |
| | bromide) (BMOP) and | Walzem R L, | |
| | DOPE | Hickman M A, | |
| | | German J B, Hansen | |
| | | R J. | |
| Cationic polysaccharides | Cationic | Published U.S. | |
| | polysaccharides | patent application | |
| | | 2002/0146826 | |
| CellFECTIN ® | 1:1.5 (M/M) | U.S. Pat. Nos. | Invitrogen (LTI) |
| | formulation of N, NI, | 5,674,908, | |
| | NII, NIII-tetramethyl- | 5,834,439 and | |
| | N, NI, NII, NIII- | 6,110,916 | |
| | tetrapalmitylspermine | | |
| | (TM-TPS) and | | |
| | dioleoyl | | |
| | phosphatidylethanola- | | |
| | mine (DOPE) | | |
| CLONfectin ™ | N-t-butyl-N′- | Ruysschaert, J. M., | BD Biosciences |
| | tetradecyl-3- | et al. (1994) | Clontech |
| | tetradecyl- | Biochem. Biophys. | |
| | aminopropion-amidine | Res. Comm. | |
| | | 203: 1622-1628 | |
| CTAB:DOPE | formulation of | | |
| | cetyltrimethyl- | | |
| | ammonium bromide | | |
| | (CATB) and | | |
| | dioleoylphosphatidyleth- | | |
| | anol-amine (DOPE) | | |
| Cytofectene | proprietary cationic | | Bio-Rad |
| | lipid and DOPE | | Laboratories |
| Cytofectin GSV | 2:1 (M/M) formulation | | (*Cytofectin GS |
| | of cytofectin GS* and | | corresponds to |
| | dioleoyl phosphatidyl- | | Gilead Sciences' GS |
| | ethanolamine (DOPE) | | 3815) |
| DC-Cholesterol (DC-Chol) | 3,β-N,(N′,N′- | | |
| | dimethylaminoethane)- | | |
| | carbamo- | | |
| | yl]cholesterol | | |
| DC-Chol:DOPE | formulation of 3,β- | Gao et al., Biochim. | |
| | N,(N′,N′- | Biophys. Res. | |
| | dimethylaminoethane)- | Comm. 179: 280-285 | |
| | carbamo- | (1991) | |
| | yl]cholesterol (DC- | | |
| | Chol) and dioleoyl | | |
| | phosphatidyl- | | |
| | ethanolamine (DOPE) | | |
| DC-6-14 | O,O′-Ditetradecanoyl- | Hum Gene Ther | |
| | N-(alpha- | 1999 Apr | |
| | trimethylammonioace- | 10; 10(6): 947-55. | |
| | tyl)diethan olamine | Development of | |
| | chloride | novel cationic | |
| | | liposomes for | |
| | | efficient gene | |
| | | transfer into | |
| | | peritoneal | |
| | | disseminated tumor. | |
| | | Kikuchi A, Aoki Y, | |
| | | Sugaya S, Serikawa | |
| | | T, Takakuwa K, | |
| | | Tanaka K, Suzuki N, | |
| | | Kikuchi H. | |
| DCPE | Dicaproylphosphtidyl- | | |
| | ethanol-amine | | |
| DDPES | Dipalmitoylphosphatid- | Behr et al. 1989. | |
| | yl-ethanolamine 5- | Efficient gene | |
| | carboxyspermylamide | transfer into | |
| | | mammalian primary | |
| | | endocrine cells with | |
| | | lipopolyamine- | |
| | | coated DNA. Proc. | |
| | | Natl. Acad. Sci. | |
| | | USA 86: 6982-6986; | |
| | | EPO Publication 0 | |
| | | 394 111 | |
| DDAB | didoceyl | | |
| | methylammonium | | |
| | bromide | | |
| Dextran and dextran | DEAE-Dextran; | J Biol Chem. 2002. | |
| derivatives or conjugates | Dextran sulfate | 277: 30208-30218. | |
| | | Efficiency of protein | |
| | | transduction is cell | |
| | | type-dependent and | |
| | | is enhanced by | |
| | | dextran sulfate. Mai | |
| | | J C, Shen H, Watkins | |
| | | S C, Cheng T, | |
| | | Robbins P D. | |
| Diquaternary ammonium salts | (examples:) N,N′- | Bioconjug Chem | Vical |
| | dioleyl-N,N,N′,N′- | 2001 Mar- | |
| | tetramethyl-1,2- | Apr; 12(2): 258-63. | |
| | ethanediamine | Diquaternary | |
| | (TmedEce), N,N′- | ammonium | |
| | dioleyl-N,N,N′,N′- | compounds as | |
| | tetramethyl-1,3- | transfection agents. | |
| | propanediamine | Rosenzweig H S, | |
| | (PropEce), N,N′- | Rakhmanova V A, | |
| | dioleyl-N,N,N′,N′- | MacDonald R C.; | |
| | tetramethyl-1,6- | U.S. Pat. No. | |
| | hexanediamine | 5,994,317 | |
| | (HexEce), and their | | |
| | corresponding N,N′- | | |
| | dicetyl saturated | | |
| | analogues (TmedAce, | | |
| | PropAce and HexAce) | | |
| DLRIE | dilauryl oxypropyl-3- | Ann N Y Acad Sci | Vical |
| | dimethylhydroxy | 1995 Nov | |
| | ethylammonium | 27; 772: 126-39. | |
| | bromide | Improved cationic | |
| | | lipid formulations | |
| | | for in vivo gene | |
| | | therapy. Felgner P L, | |
| | | Tsai Y J, Sukhu L, | |
| | | Wheeler C J, | |
| | | Manthorpe M, | |
| | | Marshall J, Cheng | |
| | | S H. | |
| DMAP | 4-dimethylamino- | | |
| | pyridine | | |
| DMPE | Dimyristoylphospatidyl- | | |
| | ethanol-amine | | |
| DMRIE | N-[1-(2,3- | Biochim Biophys | |
| | dimyristyloxy)propyl]- | Acta 1996 Jul | |
| | N,N-dimethyl-N-(2- | 24; 1312(3): 186-96. | |
| | hydroxyethyl)- | Human | |
| | ammonium bromide | immunodeficiency | |
| | | virus type-1 (HIV-1) | |
| | | infection increases | |
| | | the sensitivity of | |
| | | macrophages and | |
| | | THP-1 cells to | |
| | | cytotoxicity by | |
| | | cationic liposomes. | |
| | | Konopka K, Pretzer | |
| | | E, Felgner P L, | |
| | | Duzgunes N. | |
| DMRIE-C | 1:1 formulation of N- | U.S. Pat. Nos. | Invitrogen (LTI) |
| | [1-(2,3- | 5,459,127 and | |
| | dimyristyloxy)propyl]- | 5,264,618, to | |
| | N,N-dimethyl-N-(2- | Felgner, et al. | |
| | hydroxyethyl) | (Vical) | |
| | ammonium bromide | | |
| | (DMRIE) and | | |
| | cholesterol | | |
| DMRIE:DOPE | formulation of 1,2- | Hum Gene Ther | |
| | dimyristyloxypropyl- | 1993 Dec; 4(6): 781- | |
| | 3-dimethyl- | 8. Safety and short- | |
| | hydroxyethyl | term toxicity of a | |
| | ammonium bromide | novel cationic lipid | |
| | and dioleoyl | formulation for | |
| | phosphatidyl- | human gene therapy. | |
| | ethanolamine (DOPE) | San H, Yang Z Y, | |
| | | Pompili V J, Jaffe | |
| | | M L, Plautz G E, Xu | |
| | | L, Felgner J H, | |
| | | Wheeler C J, Felgner | |
| | | P L, Gao X, et al. | |
| DOEPC | dioleoylethylphospho- | | |
| | choline | | |
| DOHME | N-[1-(2,3- | | |
| | dioleoyloxy)propyl]- | | |
| | N-[1-(2- | | |
| | hydroxyethyl)]-N,N- | | |
| | dimethylammonium | | |
| | iodide | | |
| DOPC | dioleoylphosphatidyl- | | |
| | choline | | |
| DOPC:DOPS | 1:1 (wt %) formulation | | Avanti |
| | of DOPC | | |
| | (dioleoylphosphatidyl- | | |
| | choline) and DOPS | | |
| DOSPA | 2,3-dioleoyloxy-N-[2- | | |
| | (sperminecarboxamido | | |
| | ethyl]-N,N-di-met- | | |
| | hyl-1-propanaminium | | |
| | trifluoroacetate | | |
| DOSPA:DOPE | Formulation of 2,3- | J Gene Med 2001 | |
| | dioleoyloxy-N-[2- | Jan-Feb; 3(1): 82-90. | |
| | (sperminecarboxamido | Cationic liposome- | |
| | ethyl]-N,N-di-meth- | mediated gene | |
| | yl-1-propanaminium | transfer to rat | |
| | trifluoroacetate | salivary epithelial | |
| | (DOSPA) and dioleoyl | cells in vitro and in | |
| | phosphatidyl- | vivo. Baccaglini L, | |
| | ethanolamine (DOPE) | Shamsul Hoque A T, | |
| | | Wellner R B, | |
| | | Goldsmith C M, | |
| | | Redman R S, Sankar | |
| | | V, Kingman A, | |
| | | Barnhart K M, | |
| | | Wheeler C J , Baum | |
| | | B J. | |
| DOSPER | 1,3-Di-Oleoyloxy-2- | Buchberger et al., | Roche |
| | (6-Carboxy-spermyl)- | 1996. DOSPER | |
| | propylamid | liposomal | |
| | | transfection reagent: | |
| | | a reagent with | |
| | | unique transfection | |
| | | properties. | |
| | | Biochemica 2: 7-10. | |
| DOTAP | N-[1-(2,3- | | |
| | dioleoyloxy)propyl]- | | |
| | N,N,N-trimethyl- | | |
| | ammonium | | |
| | methylsulfate | | |
| DOTMA | N-[1-(2,3- | | |
| | dioleyloxy)propyl]- | | |
| | n,n,n- | | |
| | trimethylammonium- | | |
| | chloride | | |
| DPEPC | Dipalmitoylethylphos- | | |
| | phatidyl-choline | | |
| Effectene | (non-liposomal lipid | Histochem Cell Biol | Qiagen |
| | formulation used in | 2001 Jan; 115(1): 41- | |
| | conjunction with a | 7. Long-term | |
| | special DNA- | expression of | |
| | condensing enhancer | foreign genes in | |
| | and optimized buffer) | normal human | |
| | | epidermal | |
| | | keratinocytes after | |
| | | transfection with | |
| | | lipid/DNA | |
| | | complexes. Zellmer | |
| | | S, Gaunitz F, | |
| | | Salvetter J, Surovoy | |
| | | A, Reissig D, | |
| | | Gebhardt R. | |
| ExGen 500 | Apyrogenic solution | Ferrari S., Moro E., | Fermetas |
| | of linear 22 kDa | Pettenazzo A., Behr | |
| | polyethylenimine | J. P., Zacchello F., | |
| | (PEI) in water | Scarpa M., ExGen | |
| | | 500 is an efficient | |
| | | vector for gene | |
| | | delivery to lung | |
| | | epithelial cells in | |
| | | vitro and in vivo, | |
| | | Gene Ther, | |
| | | Oct; 4(10): 1100- | |
| | | 1106, 1997 | |
| FuGENE 6 | (proprietary | J Neurosci Methods | Roche |
| | formulation) | 1999 Oct 15; 92(1- | |
| | | 2): 145-52. Improved | |
| | | lipid-mediated gene | |
| | | transfer in C6 | |
| | | glioma cells and | |
| | | primary glial cells | |
| | | using FuGene. | |
| | | Wiesenhofer B, | |
| | | Kaufmann W A, | |
| | | Humpel C. | |
| GAP-DLRIE:DOPE | N-(3-aminopropyl)-N, | Hum Gene Ther | |
| | N-dimethyl-2,3- | 1996 Oct | |
| | bis(dodecyloxy)-1- | 1; 7(15): 1803-12. A | |
| | propaniminium | new cationic | |
| | bromide/dioleyl | liposome DNA | |
| | phosphatidylethanol- | complex enhances | |
| | amine | the efficiency of | |
| | | arterial gene transfer | |
| | | in vivo. Stephan D J, | |
| | | Yang Z Y, San H, | |
| | | Simari R D, Wheeler | |
| | | C J, Felgner P L, | |
| | | Gordon D, Nabel | |
| | | G J, Nabel E G | |
| GeneJuice | Proprietary polyamine | | Novagen |
| GeneLimo | Proprietary liposomal | | CPG, Inc. |
| | formulations of | | |
| | polycationic lipids and | | |
| | a neutral, non- | | |
| | transfecting lipid | | |
| | compound | | |
| GeneSHUTTLE ™ | Novel extruded | | |
| | DOTAP and | | |
| | cholesterol | | |
| | (DOTAP:Chol) | | |
| | formulation | | |
| Genetransfer | | | Wako Pure |
| | | | Chemical (Japan) |
| GS 2888 cytofectin | | Proc Natl Acad Sci | Gilead Sciences |
| | | USA 1996 Apr | |
| | | 16; 93(8): 3176-81. A | |
| | | serum-resistant | |
| | | cytofectin for | |
| | | cellular delivery of | |
| | | antisense | |
| | | oligodeoxynucleotides | |
| | | and plasmid | |
| | | DNA. Lewis J G, Lin | |
| | | K Y, Kothavale A, | |
| | | Flanagan W M, | |
| | | Matteucci M D, | |
| | | DePrince R B, Mook | |
| | | R A Jr, Hendren R W, | |
| | | Wagner R W. | |
| Lipofectin ® | 1:1 (w/w) formulation | U.S. Pat. Nos. | Invitrogen (LTI) |
| | of N-(1-2,3- | 4,897,355; | |
| | dioleyloxypropyl)- | 5,208,066; and | |
| | N,N,N- | 5,550,289. | |
| | triethylammonium | | |
| | (DOTMA) and | | |
| | dioleylphosphatidyleth- | | |
| | anolamine (DOPE) | | |
| LipofectACE ™ | 1:2.5 (w/w) | | Invitrogen (LTI) |
| | formulation of | | |
| | dimethyl | | |
| | dioctadecylammonium | | |
| | bromide (DDAB) and | | |
| | dioleoyl | | |
| | phosphatidylethanola- | | |
| | mine (DOPE) | | |
| LipofectAMINE ™ | 3:1 (w/w) formulation | U.S. Pat. No. | Invitrogen (LTI) |
| | of 2,3-dioleyloxy-N- | 5,334,761; and U.S. | |
| | [2(sperminecarboxami- | Pat. Nos. 5,459,127 | |
| | do)ethyl]-N,N- | and 5,264,618, to | |
| | dimethyl-1- | Felgner, et al. | |
| | propanaminium | (Vical) | |
| | trifluoroacetate | | |
| | (DOSPA) and dioleoyl | | |
| | phosphatidylethanola | | |
| | mine (DOPE) | | |
| LipofectAMINE ™ 2000 | (proprietary | | Invitrogen (LTI) |
| | formulation) | | |
| LipofectAMINE PLUS ™ | PLUS (proprietary | U.S. Pat. Nos. | Invitrogen (LTI) |
| | formulation) and | 5,736,392 and | |
| | LipofectAMINE ™ | 6,051,429 | |
| LipoTAXI ® | (proprietary | Madry H, Trippel | Stratagene |
| | formulation) | S B. Efficient lipid- | |
| | | mediated gene | |
| | | transfer to articular | |
| | | chondrocytes. Gene | |
| | | Ther. 2000 | |
| | | Feb; 7(4): 286-91. | |
| monocationic transfection | (examples:) 1-deoxy- | J Med Chem 2001 | |
| lipids | 1- | Nov | |
| | [dihexadecyl(methyl)a | 22; 44(24): 4176-85. | |
| | mmonio]-D-xylitol; 1- | Design, synthesis, | |
| | deoxy-1- | and transfection | |
| | [methyl(ditetradecyl)am- | biology of novel | |
| | monio]-D-arabinitol; | cationic glycolipids | |
| | 1-deoxy-1- | for use in liposomal | |
| | [dihexadecyl(methyl)am- | gene delivery. | |
| | monio]-D-arabinitol; | Banerjee R, | |
| | 1-deoxy-1- | Mahidhar Y V, | |
| | [methyl(dioctadecyl)am- | Chaudhuri A, Gopal | |
| | monio]-D-arabinitol | V, Rao N M. | |
| O-Chol | 3 beta[1- | Gene Ther 2002 | |
| | ornithinamide- | Jul; 9(13): 859-66. | |
| | carbamoyl] cholesterol | Intraperitoneal gene | |
| | | delivery mediated | |
| | | by a novel cationic | |
| | | liposome in a | |
| | | peritoneal | |
| | | disseminated | |
| | | ovarian cancer | |
| | | model. Lee M J, Cho | |
| | | S S, You J R, Lee Y, | |
| | | Kang B D, Choi J S, | |
| | | Park J W, Suh Y L, | |
| | | Kim J A, Kim D K, | |
| | | Park J S. | |
| OliogfectAMINE ™ | (proprietary | | Invitrogen (LTI) |
| | formulation) | | |
| Piperazine based amphilic | Piperazine based | U.S. Pat. Nos. | Vical |
| cationic lipids | amphilic cationic | 5,861,397 and | |
| | lipids | 6,022,874 | |
| PolyFect | (activated-dendrimer | | Qiagen |
| | molecules with a | | |
| | defined spherical | | |
| | architecture) | | |
| Protamine | Protamine mixture | Gene Ther 1997 | Sigma |
| | prepared from, e.g., | Sep; 4(9): 961-8. | |
| | salmon, salt herring, | Protamine sulfate | |
| | etc.; can be supplied | enhances lipid- | |
| | as, e.g., a sulfate or | mediated gene | |
| | phosphate. | transfer. Sorgi F L, | |
| | | Bhattacharya S, | |
| | | Huang L. | |
| SuperFect | (activated-dendrimer | Tang, M. X., | Qiagen |
| | molecules with a | Redemann, C. T., | |
| | defined spherical | and Szoka, Jr., F. C. | |
| | architecture) | (1996) In vitro gene | |
| | | delivery by | |
| | | degraded | |
| | | polyamidoamine | |
| | | dendrimers. | |
| | | Bioconjugate Chem. | |
| | | 7: 703; published | |
| | | PCT applications | |
| | | WO 93/19768 and | |
| | | WO 95/02397 | |
| Tfx ™ | N,N,N′,N′- | | Promega |
| | tetramethyl-N,N′- | | |
| | bis(2-hydroxyethyl)- | | |
| | 2,3-di(oleoyloxy)-1,4- | | |
| | butanediammonium | | |
| | iodide] and DOPE | | |
| TransFAST ™ | N,N [bis (2- | | Promega |
| | hydroxyethyl)-N- | | |
| | methyl-N-[2,3- | | |
| | di(tetradecanoyloxy) | | |
| | propyl] ammonium | | |
| | iodide and DOPE | | |
| TransfectAce | | | Invitrogen (LTI) |
| TRANSFECTAM ™ | 5-carboxylspermyl- | Behr et al. 1989. . | Promega |
| | glycine | Proc. Natl. Acad. | |
| | dioctadecylamide | Sci. USA 86: 6982- | |
| | (DOGS) | 6986; EPO | |
| | | Publication 0 394 | |
| | | 111 | |
| TransIT ® -LT1 and | Proprietary | | Panvera, Mirus |
| TransIT ® -LT2 | combination of a | | |
| | nontoxic cellular | | |
| | protein & a | | |
| | proprietary polyamine | | |
| TransMessenger | (lipid-based | | Qiagen |
| | formulation that is | | |
| | used in conjunction | | |
| | with a specific RNA- | | |
| | condensing enhancer | | |
| | and an optimized | | |
| | buffer; particularly | | |
| | useful for mRNA | | |
| | transfection) | | |
| Vectamidine | 3-tetradecylamino-N- | FEBS Lett 1997 Sep | |
| | tert-butyl-N′- | 8; 414(2): 187-92. | |
| | tetradecylpropionami- | The role of | |
| | dine (a.k.a. diC14- | endosome | |
| | amidine) | destabilizing activity | |
| | | in the gene transfer | |
| | | process mediated by | |
| | | cationic lipids. El | |
| | | Ouahabi A, Thiry | |
| | | M, Pector V, Fuks | |
| | | R, Ruysschaert J M, | |
| | | Vandenbranden M. | |
| X-tremeGENE Q2 | (proprietary | | Roche Molecular |
| | formulation) | | Biochemicals |

All patents, patent publications, patent applications and other published references mentioned herein are hereby incorporated by reference in their entirety as if each had been individually and specifically incorporated by reference herein

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Binding of Slyd Protein to a Biarsenical Compound

The mode of binding of a biarsenical to a target sequence was examined using the Expressway™ in vitro protein synthesis kit (Invitrogen, Carlsbad, Calif.) and SDS-PAGE. Following the manufacturer's protocol, 1 μg of vector DNA encoding SlyD+His tag (SEQ ID NO.:4), SlyD-C167A/C168A (SEQ ID NO.:5), or SlyD-truncl7l (SEQ ID NO.:6) was added to a total volume of 50 μL of S30 E. coli extract and reaction buffer. The reaction was placed at 37° C. with 225 rpm shaking for two hours. Then, 5 μL of RNase A was added to the reaction, and the reaction was incubated for 15 minutes at 37° C. Protein synthesis reactions were prepared for SDS-PAGE analysis by an acetone precipitation procedure. In brief, 5 μl of reaction was added to 20 μL of 100% acetone. After mixing well, the mixture was centrifuged for 5 minutes at room temperature in a microcentrifuge at 12,000 rpm. The supernatant was removed and the pellet was dried for 5 minutes. The pellet was resuspended in 50 μL of LDS sample buffer (Invitrogen, Carlsbad, Calif.) containing 10 μM FlAsH-EDT₂. The samples were heated at 70° C. for 10 minutes. 10 μL of the samples then were loaded onto a 4-12% NUPAGE® pre-cast gel (Invitrogen) using MES running buffer. The gel was electrophoresed at 200 volts for about 30 minutes. Immediately following electrophoresis, the gel was removed from the cassette and visualized on a UV light box.

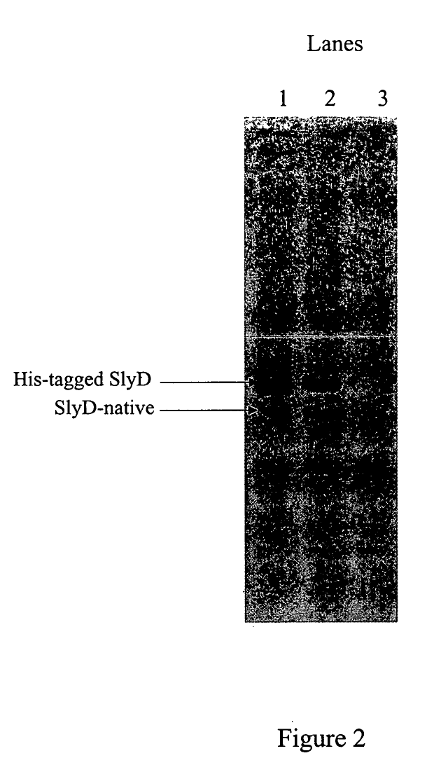

The results (FIG. 2) indicate that biarsenical labeling of SlyD-trunc 171 (lane 3) is substantially reduced if not completely eliminated, relative to SlyD+His tag (lane 1) or SlyD-C167A/C168A (lane 2). This identifies the biarsenical-binding region of the SlyD protein, which is present in wildtype SlyD, unaltered in C167A/C168A and missing in SlyD-trunc 171, having the sequence:

GHDHGHEHGGEGCCGGKGNGGCGCH. (SEQ ID NO.:7)

A derivative of this sequence contains 4 cysteine residues and thus likely represents the minimal biarsenical-binding site sequence:

CCGGKGNGGCGC (SEQ ID NO.:2)

### Example 2

### IVPS Extracts from SLYD Mutant Cells

The following protocols are used to prepare S30 extracts.

2.A. Cell Paste:

Cells are grown in 50-L Buffered 2X YT (Tryptone, 16 g/L; Yeast Extract, 10 g/L; Sodium chloride, 5 g/L; Dibasic sodium phosphate anhydrous Na2HPO4, 5.68 g/L; Monobasic sodium phosphate anhydrous NaH2PO4, 2.64 g) supplemented with Cerelose (5 g/L). Cells are incubated at 37° C. on a rotating platform (typically, 250 rpm), until the OD590 reaches a range of from about 3.0 to about 5.0, which typically takes from about 6 to about 8 h. The cells are freshly inoculated into fresh media with a starting OD590 of about 0.05 to about 0.10, and then incubated at 37° C., at 250 rpm, 50 slpm, 5 psi, to an OD590 of from about 3.0 to about 3.5. Cells are transferred to Sorvall GS3 bottles and ecntrifuged for 15 min at 5000× g. The supernatant is removed, with aspiration if needed. The cell paste can be stored, preferably for 5 days or less, at −80° before proceeding to the next step.

One gram of cell paste, thawed first if stored at −80° C., is resuspended in 1 ml of chilled (4° C.) S30 buffer with DTT added immediately prior to use (for example, 250 ml S30 buffer for 250 g cells). Swirl the cells gently by hand for a few minutes (without generating froth) to hasten the resuspension process. Place a sterile stir bar into bottle containing cells and stir gently for approximately 15 min to completely resuspend cells. Place on ice immediately. Do not add any more buffer, as volume is critical to final total protein concentration of extract.

The cells are swirled gently by hand for a few minutes (without generating froth) to hasten the resuspension process. A sterile stir bar is placed into a bottle containing cells and is stirred gently for approximately 15 min to completely resuspend cells. The resuspension is placed on ice immediately.

2.B. Cell Lysis:

The resuspended cells are washed with 20 volumes of S30 buffer, 1 mM DTT. This is carried out by adding S30, 1 mM DTT to the cells and “mashing and stiring” with a 25 ml pipette until the cell paste is completely dissolved. The suspension is spun in an RC3B centrifuge for 20 minutes at 4,500 RPM. The supernatant is decanted, and the wash is repeated.

The resuspended cells are poured in sterile 1 L side-arm flask. The pouring is done gently and, if particulate matter is present, can be filtered through a piece of sterile cheesecloth as it is poured into the flask.

The side-arm flask containing the cells is attached to a vacuum pump, and cells are de-gassed for approximately 15 min. The cells are swirled occasionally to promote degassing. Once the cells are degassed, care is taken to not swirl the solution or generate bubbles.

Five (5) ml resuspended cells is placed into 995 ml water (1:200 dilution) to determine a starting OD. This sample is vortexed and read at 590 nm using water as a blank. Immediately before proceeding to cell disruption, 0.1 M Phenylmethanesulfonyl fluoride (PMSF) is added to the degassed, resuspended cell paste. Five (5) μl of 0.1 M PMSF per ml of cell suspension is used.

An Emulsiflex C50 homogenizer (Avestin Inc., Ottawa, Canada) is used to disrupt the cells. Preferably, the homogenizer is chilled for at least about 1 h before use. The compressed air outlet is turned to 115-120 psi, and the timer set is to 60 min. The homogenizing pressure is set to 25,000 psi. The regulator knob is set to a reading of 80-85-100 psi. A sterile 0.5 L container is placed at the outlet receiving reservoir. The inlet reservoir is filled with the de-gassed and filtered cell suspension. The homogenizer is started. Pressure is kept at from least about at 25,000 to about 30,000 psi.; the homogenizer may stall if the pressure exceeds 30,000 psi. It should take approximately 15-20 min to pass 500 ml cell suspension through the homogenizer.

The efficiency of lysis should be greater than about 90%. If less then 90%, the cell suspension is passed through the homogenizer again. The efficiency of lysis is calculated as follows (First Pass OD590/initial OD590, see above)×100=% not lysed; 100− % not lysed=% efficiency of lysis.

One (1) M DTT is immediately added to lysate to a final concentration of 1 mM (e.g., 250 ml 1 M DTT per 250 ml lysate). The lysate is then centrifuged at 16,000 rpm (30,000× g) in an SS34 rotor for 40 min at 4° C. The upper four-fifths of supernatant is removed with a sterile plastic graduated pipet and collect in a sterile 1 L container. Care is taken to not pour off the supernatant because the pellet is very loose. Care is taken to avoid any cloudy precipitate near the pellet.

The volume of supernatant is measured. Preferably, the volume (in ml) will be approximately the same as the weight of starting material (e.g., for 50 g cells, the volume of supematant is ˜50 ml). Five (5) ml of pre-incubation mix is added per 25 ml supernatant (e.g., 250 ml supernatant will require 50 ml pre-incubation mix). The mixture is incubated in a 37° C. shaking water bath, shaking gently at 150 rpm for 80 min. Care is taken so as to not shake the solution enough to form bubbles.

2.C. Preincubation:

The volume of Pyruvate Kinase (Sigma P7768) 54421 required for the Pre-Incubation Mix (PIM) is calculated as follows. The manufacturer's specifications permit this product to be within the range of from about 1200 to about 2500 U/ml. The following formulas are used to calculate to what volume to add for a final concentration of 10.08 U/ml (e.g., 756 U in 75 ml).
10.08 U/ml×Volume of PIM to prepare_ml=X
X/Concentration of Pyruvate Kinase_U/ml=_ml of pyruvate kinase required for PIM.

The PIM is prepared by adding the components in Table 3 in the order listed. The PIM is prepared just before use and stored on ice.

TABLE 3
PRE-INCUBATION MIX (PIM)

| Final Conc. | | Component |
|---|---|---|
| — | | Gibco Water |
| 0.44 | M | Tris-Acetate, pH 8.2 at 22 C. |
| 13.8 | mM | Magnesium Acetate |
| 20 | mM | ATP pH 7.0 |
| 126 | mM | Phosphoenol Pyruvate pH 7.0 |
| 6.6 | mM | DTT |
| 60 | μM | Amino Acid Mix (-Met) |
| 60 | μM | Methionine |

Phosphoenol Pyruvate (PEP) can be prepared as either a monosodium salt or monopotassiom salt:

Phosphoenyl Pyruvate-Monosodium Salt (Roche): 3.12 g is added to 2.5 ml of water (Gibco) in a sterile 50ml conical tube. The tube is placed on ice and 2.5 ml 10N KOH is added, and the solution is mixed well. Using a clean RNase/DNase free probe, the pH of the solution is adjusted to 7.0+0.2 using drops of KOH. Because the pH of the solution will change rapidly, care is taken to be conservative with the KOH after pH 6.5 is reached. If the pH is overshot, the solution must be discarded. The final volume should be about 10 ml; water is added if necessary to reach the required volume. This solution can be stored at −20° C. for up to about 1 month.

Phosphoenyl Pyruvate-Monopotassium Salt (Roche):

3.09 g is added to 2.5 ml of water (Gibco) in a sterile 50ml conical tube. The tube is placed on ice and 2.5 ml 10N KOH is added, and the solution is mixed well. Using a clean RNase/DNase free probe, the pH of the solution is adjusted to 7.0+0.2 using drops of KOH. Because the pH of the solution will change rapidly, care is taken to be conservative with the KOH after pH 6.5 is reached. If the pH is overshot, the solution must be discarded. The final volume should be about 10 ml; water is added if necessary to reach the required volume. This solution can be stored at −20° C. for up to about 1 month.

Five (5) ml of PIM per 25 ml of supernatant is added (e.g., 250 ml of supernatant will require 50 ml pre-incubation mix). Care is taken to calculate the volume of the mix, because too much mix will dilute the extract, which may reduce its activity. The container is placed into a 37° C. shaker, and shook gently (e.g., at 150 rpm) for 2 h. Care is taken to make sure that the solution is not shaking fast enough to form bubbles.

2.D. Dialysis:

The solution is dialyzed 3×45 min with 50 volumes of S30 buffer (containing DTT) at 4° C. (e.g., 250 ml lysate is dialyzed in 12.5 L S30 buffer per change). Dialysis tubing with a molecular weight exclusion limit of 12,000 to 14,000 daltons is used, and is rinsed well with distilled water just prior to use.

The dialyzed material is poured into sterile, dedicated SS-34 centrifuge tubes, and centrifuged at 4,000 rpm (3000× g) with the SS-34 and rotor for 12 min at 4° C. The supernatant is removed using a sterile plastic graduated pipette, and is not poured off because the pellet is very loose. It is then immediately placed on ice.

The supernatant is mixed well by gently swirling and is distribute in 25 ml aliquots in 50 ml conical tubes. The ahquots are frozen in liquid nitrogen using a Cyromed. Alternatively, aliquots are frozen by submerging them in dry ice for 30 min. The extract is stored at −80° C.

The following day, an aliquot is thawed and its protein content is determined using a Bradford assay. The total protein should be from about 25 to about 50 mg/ml, preferably from about 28 to about 42 mg/ml.

Preparation of S30 extracts from another slyD strain, A19 slyD::kan, is described in U.S. Provisional Patent Application No. 60/587,583, filed Jul. 14, 2004, which is hereby incorporated by reference. The A19 slyD::kan strain requires 50 mg/ml kanamycin antibiotic during 6-8 hour and overnight growth incubations, but this is optional during fermentation.

### Example 3

### Reagents for IVPS

3.A. Amino Acid Mixtures:

For IVPS reactions, amino acid mixtures were prepared according to the following procedure. All of the amino acid components are included in the final amino acid mix, which will contain a final concentration of 50 mM for each component. All amino acids used in the preparation were ordered as a single unit of powdered material from Sigma. Amino acids were added in the order written in Table 4, below.

TABLE 4
COMPONENTS OF AMINO ACID MIXTURES

| | | Amount to make 100 |
|---|---|---|
| Amino Acid | M.W (g/mol) | ml of 50 mM mix |
| Alanine | 89.1 | 0.45 g |
| Arginine | 174.2 | 0.87 g |
| Asparagine | 150.1 | 0.75 g |
| Aspartate (Aspartic acid) | 133.1 | 0.67 g |
| Cysteine | 121.2 | 0.61 g |
| Glutamate (Glutamic acid) | 147.1 | 0.74 g |
| Glutamine | 146.1 | 0.73 g |
| Glycine | 75.1 | 0.38 g |
| Histidine | 155.2 | 0.78 g |
| Isoleucine | 131.2 | 0.66 g |
| Leucine | 131.2 | 0.66 g |
| Lysine | 182.7 | 0.91 g |
| Methionine | 149.2 | 0.74 g |
| Phenylalanine | 165.2 | 0.83 g |
| Proline | 115.1 | 0.58 g |
| Serine | 105.1 | 0.53 g |
| Threonine | 119.1 | 0.60 g |
| Tryptophan | 204.2 | 1.00 g |
| Tyrosine | 181.2 | 0.91 g |
| Valine | 117.2 | 0.59 g |

The first component is weighed accurately to +0.01 g and added into an appropriately sized sterile container with a screw cap lid. The weighing procedure is repeated for the next component, which is then added to the container; this continues until all 20 amino acids are weighed and added to the sterile container. Once all 20 components are combined, Gibco water is added to a final volume of 100 ml.

The mixture is placed on a Labquake (Bamstead International, Dubuque, Iowa) and gently rocked to get as much of the mix into solution as possible (˜30 to 120 min at room temperature). The final mix is a slurry, not a completely dissolved solution.

The slurry is aliquoted into sterile 50-ml Falcon tubes at 20 ml/container. Care is taken to ensure that the slurry is well mixed so that the insoluble components are evenly distributed immediately before aliquotting. The slurry should not be aliquoted if it has been settling for more than 10 s without stirring. The amino acid mix can be stored at −20° C. for up to 2 years.

Table 5 lists the set of 20 naturally occurring amino acids commonly found in proteins, the one and three letter codes associated with each amino acid, and the corresponding codons that encode each amino acid.

TABLE 5
NATURALLY OCCURRING AMINO ACIDS
AND CODONS THAT ENCODE THEM

| | 3-Letter | 1-Letter | |
|---|---|---|---|
| Full name | Code | Code | Standard Codons* |
| Alanine | Ala | A | GCU, GCC, GCA, GCG |
| Arginine | Arg | R | CGU, CGC, CGA, CGG, |
| | | | AGA, AGG |
| Asparagine | Asn | N | AAU, AAC |
| Aspartic Acid | Asp | D | GAU, GAC |
| Cysteine | Cys | C | UGU, UGC |
| Glutamine | Gln | Q | CAA, CAG |
| Glutamic Acid | Glu | E | GAA, GAG |
| Glycine | Gly | G | CGU, CGC, CGA, CGG |
| Histidine | His | H | CAU, CAC |
| Isoleucine | Ile | I | AUU, AUC, AUA |
| Leucine | Leu | L | UUA, UUG, CUU, CUC, |
| | | | CUA, CUG |
| Lysine | Lys | K | AAA, AAG |
| Methionine | Met | M | AUG |
| Phenylalanine | Phe | F | UUU, UUC |
| Proline | Pro | P | CCU, CCC, CCA, CCG |
| Serine | Ser | S | UCU, UCC, UCA, UCG, |
| | | | AGU, AGC |
| Threonine | Thr | T | ACU, ACC, ACA, ACG |
| Tryptophan | Trp | W | UGG |
| Tyrosine | Tyr | Y | UAU, UAC |
| Valine | Val | V | GUU, GUC, GUA, GUG |

*Codons are depicted in this table as they appear in mRNA. Corresponding codons in DNA molecules would substitute a thymidine (T) nucleotide for any uracil (U) nucleotide in the RNA sequence.

3.B. IVPS Buffer

Expressway™ Plus with Lumio™ Technology 2.5×IVPS reaction buffer.

TABLE 6
2.5× IVPS BUFFER (MINUS AMINO ACIDS)

| Component | 2.5× Concentration | 1× Concentration | |
|---|---|---|---|
| 1 M HEPES-KOH pH 7.6 | 145 | 58 | mM |
| 1 M DTT | 4.25 | 1.7 | |
| 100 mM ATP | 3 | 1.2 | |
| 100 mM UTP | 2.2 | 0.88 | |
| 100 mM CTP | 2.2 | 0.88 | |
| 100 mM GTP | 2.2 | 0.88 | |
| 20 mg/ml Folinic Acid | 85 | 34 | ug/ml |
| 2 M Acetyl Phosphate | 75 | 30 | |
| 3 M Potassium Glutamate | 575 | 230 | |
| 2 M MgOAc | 30 | 12 | |
| 7.5 M NH₄OAc | 200 | 80 | |
| 100 mM cAMP | 1.625 | 0.65 | |
| 1.5 M PEP mono Sodium | 75 | 30 | |
| 50% PEG | 5 | 2 | |
| Molecular Biology Grade Water | | | |

TABLE 7
IVPS FINAL CONCENTRATIONS

| Component | Final Concentration | |
|---|---|---|
| HEPES (pH 7.5) | 57 | mM |
| DTT | 1.76 | mM |
| Sodium ATP | 1.2 | mM |
| Sodium CTP | 0.86 | mM |
| Sodium GTP | 0.86 | mM |
| Sodium UTP | 0.86 | mM |
| Folinic Acid | 34 | ug/ml |
| Acetyl Phosphate | 30 | mM |
| Potassium Glutamate | 230 | mM |
| Ammonium Acetate | 80 | mM |
| Magnesium Acetate | 12 | mM |
| cAMP | 0.66 | mM |
| PEP/K | 30 | mM |

### Example 4

### In Vitro Protein Synthesis in Extracts from SLYD Mutant Cells

This example demonstrates a system for the rapid detection of protein products using Lumio™ Technology. The Expressway™ Plus system was employed using cell extracts made from E. coli slyD mutant strain JDP689. Using extracts from this strain reduces non-specific binding of the Lumio™ Detection Reagent to endogenous SlyD protein, providing an optimal background for detection of tetracysteine-tagged proteins.

For standard Expressway™ Plus protein synthesis reactions, 4 μl DNase/RNase-free water, 20 μl 2.5×IVPS Plus E. coli Reaction Buffer, 1 μl T7 RNA polymerase, and 20 μl IVPS Plus E. coli extract (from slyD mutant E. coli strain JDP689) were pre-mixed in 2-ml tubes on ice. One microgram of DNA templates was added and the final volume of the reaction brought to 50 μl with water and mixed thoroughly. Reactions were incubated at 37° C. for 2 hours in a thermomixer or placed in 96-well plates in the fluorometer. Adenylate kinase was produced in vivo.

Gel samples were prepared by precipitating 5 μl of the 50-μl reactions into 20 μl 100% acetone and incubated at +4° C. for >20 minutes. Reactions were centrifuged at maximum speed in a microcentrifuge for 5 minutes, acetone was aspirated, and the pellets were resuspended in 50 μl of 1×Lumio™Green Detection Reagent. One microliter of this material was loaded onto 4-12% NuPAGE® gradient gels. Gels were visualized using a UV light box or Typhoon 8600 Variable mode Imager. For total protein profiling, gels were post-stained with Coomassie® brilliant blue.

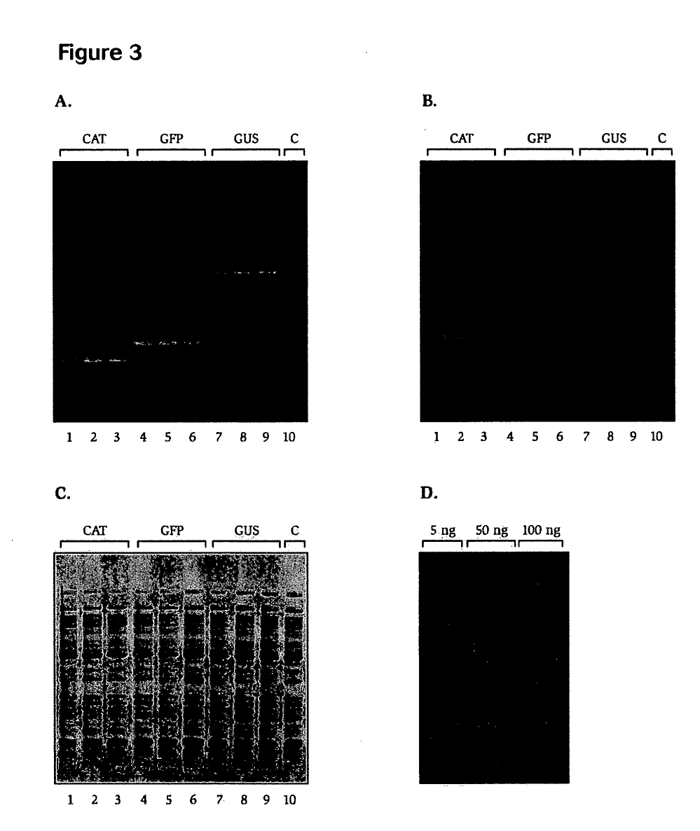

To test the ability of the Lumio™ Reagent to detect proteins synthesized with the Expressway™ Plus system, tetracysteine-tagged chloramphenicol acetyltranferase (CAT), green fluorescent protein (GFP), and glucoronidase (GUS) were expressed in vitro. The expressed proteins were then labeled with the Lumio™ Green Detection Reagent, separated by electrophoresis, and imaged with both a Typhoon laser gel scanner and a standard UV light box (FIGS. 3A and 3B). The tetracysteine-tagged proteins stand out against a low background signal in both images. Comparing the results of the fluorescent images to the total protein profile (FIG. 3C) illustrates the sensitivity and ease of detection using the Lumio™ Reagent with the Lumio™ sequence. FIG. 3D demonstrates that as little as 5 ng of purified Adenylate kinase 1 can be easily detected in-gel using the Lumio™ Detection Reagent. In contrast to modified amino acid methods, the consistent 1:1 stoichiometry of the Lumio™ Reagent binding to tetracysteine sequences results in uniform labeling of proteins.

### Example 5

### Real-Time Detection of Protein Expression

Real-time incorporation of the Lumio™ sequence was measured directly from 50-μl IVPS reactions with 20 μM Lumio™ Green Detection Reagent in a 96-well plate at 37° C. using a Molecular Devices Spectra Max GeminiXS plate reader. The excitation wavelength was set at 500 nm, while emission was monitored at 535 nm. Readings were collected at 10-minute intervals over a 2-hour incubation period. Real-time monitoring of GFP production was performed in a similar manner without the addition of Lumio™ Green Detection Reagent.

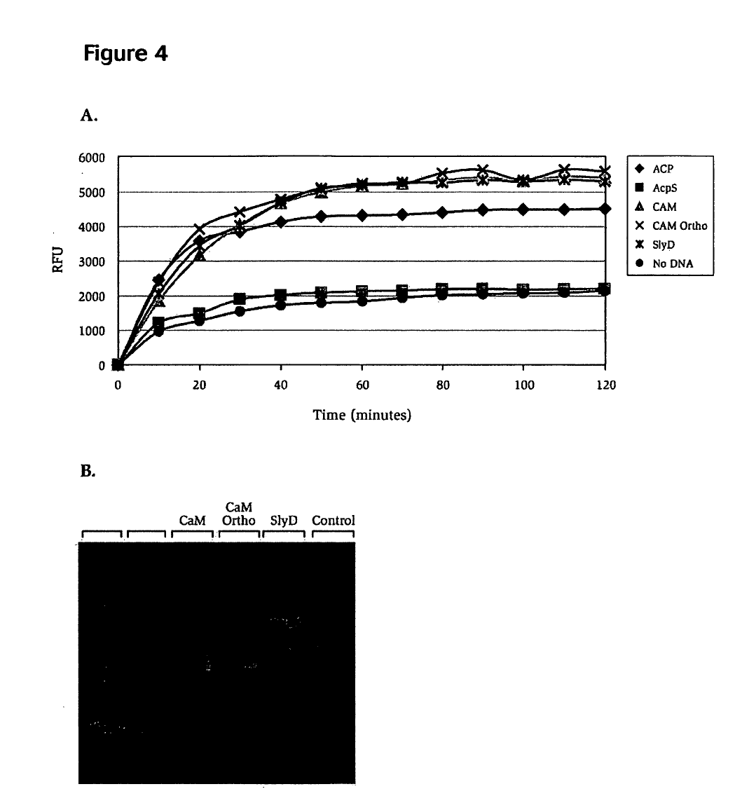

Monitoring real-time protein production was performed directly in cell-free extract reactions with Lumio™ technology. Lumio™ Green Detection Reagent was added to IVPS reactions prior to 37° C. incubation. As the reaction proceeded, an increase in fluorescence was monitored with a standard fluorometer (FIG. 4A). Approximately 2-3 fold increase in fluorescence of the Lumio™ sequence was observed compared to a vector DNA control. In-gel detection of the co-translationally monitored proteins is shown for comparison in FIG. 4B.

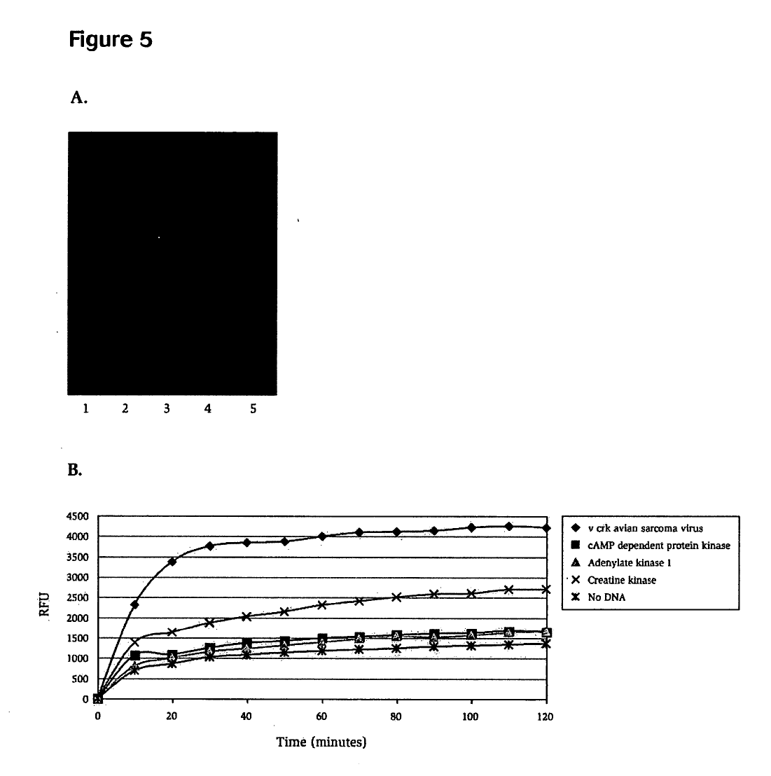

The detection of proteins expressed using the Expressway™ Plus System with Lumio™ Technology was also demonstrated using Gateway® Technology and the Ultimate™ Human ORF clone collection. Cysteine tagged ORF clones were detected in gel and monitored in real-time with Lumio™ Detection Reagent (FIG. 5).

### Example 6

### IVPS Extracts Comprising Detergents

This Example describes IVPS systems, which may be prepared from SlyD-deficient cells, that are supplemeneted with one or more detergents. Preferred detergents are non-ionic and zwitterionic detergents. A particularly preferred detergent is Triton X-100.

The method for preparing an S30 extract is described in Example 2, supra. This procedure was carried out but with the following modifications:

Lysis with Detergents:

Variation of protocol of Example 2.B.

The cell paste was resuspended in Detergent resuspension buffer by adding S30 (+DTT), 0.1% TX-100 (from a 10% TX-100 solution protein grade, Calbiochem) buffer to the cell paste, 1 ml per each gram of cell paste. Care was taken to not add more buffer, as the volume is critical to the final protein concentration of extract “mash and stir” with a 25 ml pipette until cell paste is completely dissolved. The temperature of the mixture was held near 4° C. by placing it in an ice bucket if necessary.

Additional or alternative detergents have been added at this step. Detergents which perform well in the extract include without limitation CHAPs (about 1%), Brij35 (about 0.1%), zwittergent3-14 (about 0.1%), Brij 58P (about 0.1%), n-Dodecyl-B-D-maltoside (about 0.1%).

Other detergents may be used. For a non-limiting list of detergents that may be used int the invention, see http://psyche.uthct.edu/shaun/SBlack/detergnt.html.

Dialysis with or without Detergent:

Variation of protocol of Example 2.D.

After incubation, the extract was placed into dialysis against S30 (+DTT) +0.1% Triton X100 (or other detergent) with a stir bar at 4° C. for 2 h.

After 2 h, the extract was transferred into fresh dialysis buffer and was dialyzed against at least 50×volumes (of 9.29) of S30 (+DTT) +0.1% Triton X100 (or detergent of choice) with stir bar at 4° C. overnight.

The choice of whether or not to include or omit detergent in the dialysis buffer depends in part on critical micelle concentration. Triton X-100, for example, forms relatively large micelles at a relatively low concentration, and is preferably omitted from the dialysis buffer.

IVPS Extracts Lysed With Detergent:

IVPS system made from detergent-inclusive lysates produce more soluble protein (better yield). Without wishing to be bound by any particular theory, this could result from processes such as the release of chaperone proteins from the cell membrane and/or the molecules binding detergent to protein molecules.

1. A cellular extract from an organism comprising a SlyD gene, substantially free of a SlyD polypeptide that binds to a bi-arsenical reagent.

2. The cellular extract of claim 1, wherein said cellular extract is competent for IVTT.

3. The cellular extract of claim 1, wherein the cellular extract is a bacterial extract.

4. The cellular extract of claim 1, wherein the cellular extract is an E. coli extract.

5. The cellular extract of claim 1, wherein the cellular extract is from an E. coli comprising a SlyD mutant gene.

6. The cellular extract of claim 5, wherein the SlyD mutant gene encodes a SlyD that is mutated in an amino acid sequence that binds a biarsenical molecule.

7. The cellular extract of claim 5, wherein the SlyD mutant gene encodes a truncated SlyD protein.

8. The cellular extract of claim 1, further comprising a nuclease inhibitor.

9. The cellular extract of claim 8, wherein the nuclease inhibitor is a Gam protein.

10. The cellular extract of claim 1, wherein the extract has reduced activity of at least one enzyme that catalyzes hydrolysis of high energy phosphate bonds or hydrolysis or formation of phosphodiester bonds.

11. The cellular extract of claim 1, further comprising at least one inhibitor of at least one enzyme that catalyzes hydrolysis of high energy phosphate bonds or hydrolysis or formation of phosphodiester bonds.

12. The cellular extract of claim 1, further comprising at least two energy sources providing chemical energy for synthesis.

13. The cellular extract of claim 1, further comprising a nucleic acid encoding a fusion protein encoding an exogenous protein fused to a tag.

14. The cellular extract of claim 13, wherein the tag binds a biarsenical reagent.

15. A kit for the in vitro production of proteins, said kit comprising a cellular extract according to claim 1.

16. The kit of claim 14, further comprising one or more packaged solutions selected from the group consisting of a buffer, a solution comprising magnesium, a solution comprising amino acids and a solution comprising ribonucleotide triphosphates.

17. The kit of claim 16, wherein the buffer is an in vitro transcription/translation buffer.

18. A method for producing a protein comprising combining a nucleic acid that encodes said protein with the cellular extract of claim 1.

19. The method of claim 16, wherein the nucleic acid encodes a fusion protein comprising a tag.

20. The method of claim 19, wherein the tag binds a biarsenical reagent.

21. The method of claim 20, further comprising contacting the protein with a biarsenical reagent.

22. The method of claim 21, further comprising detecting the biarsenical reagent.

23. The method of claim 22, wherein the detection is carried out in real-time.

24. A method for producing a protein comprising combining a nucleic acid that encodes said protein with the cellular extract of claim 1.

25. A polypeptide comprising an amino acid sequence that binds that specifically binds to EDT₂[4′,5′-bis(1,3,2-dithioarsolan-2-yl)fluorescein-(1,2-ethanedithiol)₂, selected from the group consisting of CCGGKGNGGCGC (SEQ ID NO. 2), CCGGHGHDHGHEHGGEGCCGGKGNGGCGC (SEQ ID NO. 3) and AAGGHGHDHGHEHGGEGCCGGKGNGGCGC (SEQ ID NO. 8).

26. A fusion protein comprising an amino acid sequence that binds that specifically binds to EDT₂[4′,5′-bis(1,3,2-dithioarsolan-2-yl)fluorescein-(1,2-ethanedithiol)₂, selected from the group consisting of CCGGKGNGGCGC (SEQ ID NO. 2), CCGGHGHDHGHEHGGEGCCGGKGNGGCGC (SEQ ID NO. 3), and AAGGHGHDHGHEHGGEGCCGGKGNGGCGC (SEQ ID NO. 8).

27. A nucleic acid encoding an amino acid sequence that binds that specifically binds to EDT₂[4′,5′-bis(1,3,2-dithioarsolan-2-yl)fluorescein-(1,2-anedithiol)₂, selected from the group consisting of CCGGKGNGGCGC (SEQ ID NO. 2), CCGGHGHDHGHEHGGEGCCGGKGNGGCGC (SEQ ID NO. 3) and GGHGHDHGHEHGGEGCCGGKGNGGCGC (SEQ ID NO. 8).


SEQUENCE LISTING (12 sequences)

SEQ ID NO: 1 - protein, 196 aa, Escherichia coli
MKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 2 - protein, 12 aa, Artificial
  SlyD tetracysteine sequence
CCGGKGNGGCGC

SEQ ID NO: 3 - protein, 29 aa, Artificial
  SlyD hexacysteine sequence
CCGGHGHDHGHEHGGEGCCGGKGNGGCGC

SEQ ID NO: 4 - protein, 229 aa, Artificial
  SlyD and His tag
MRGSHHHHHHGMASMTGGQQMGRDLYDDDDKDPMKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 5 - protein, 229 aa, Artificial
  SlyD C167A/C168A
MRGSHHHHHHGMASMTGGQQMGRDLYDDDDKDPMKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGAAGGHGHDHGHEHGGEGCCGGKGNGGCGCH

SEQ ID NO: 6 - protein, 204 aa, Artificial
  SlyD trunc171
MRGSHHHHHHGMASMTGGQQMGRDLYDDDDKDPMKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGH

SEQ ID NO: 7 - protein, 25 aa, Artificial
  Biarsenical labeling of SlyD-trunc171
GHDHGHEHGGEGCCGGKGNGGCGCH

SEQ ID NO: 8 - protein, 29 aa, Artificial
  Polypeptide that binds to EDT2[4′,5′-bis(1,3,2-dithioarsolan-2-yl)fluorescein-(1, 2-ethanedithiol)2
AAGGHGHDHGHEHGGEGCCGGKGNGGCGC

SEQ ID NO: 9 - protein, 6 aa, Artificial
  Acyl carrier protein expressed from pRSETb
CCPGCC

SEQ ID NO: 10 - protein, 6 aa, Artificial
  N terminal of CaM expressed from pRSETb
CCEQCC

SEQ ID NO: 11 - protein, 8 aa, Artificial
  C terminal of CaM Ortho expressed in pRSETb
CGPCCGPC

SEQ ID NO: 12 - protein, 6 aa, Artificial
  Arsenic binding domain
CCXXCC